(19) United States
(12) Patent Application Publication
(10) Pub. No.: US 2010/0137206 A1
(43) Pub. Date: Jun. 3, 2010

(54) NOVEL LIGAND GUIDED BLOCK COPOLYMERS FOR TARGETED DRUG DELIVERY

(75) Inventors: Afsaneh Lavasanifar, Edmonton (CA); Xiao-Bing Xiong, Edmonton (CA)

Correspondence Address: BENNETT JONES LLP;C/O MS ROSEANN CALDWELL, 4500 BANKERS HALL EAST, 855 - 2ND STREET, SW, CALGARY, AB, T2P 4K7, CA

(73) Assignee: THE GOVERNORS OF THE UNIVERSITY OF ALBERTA, Edmonton, AB (CA)

(21) Appl. No.: 12/517,659

(22) PCT Filed: Dec. 17, 2007
(86) PCT No.: PCT/CA2007/002298

§ 371 Date: Dec. 18, 2009

Related U.S. Application Data
(60) Provisional application No. 60/870,330, filed on Dec. 15, 2006.

(51) Int. Cl. A61K 31/704; A61K 31/7088; A61K 38/16; A61K 38/02; C08G 63/08

(52) U.S. Cl. 514/12; 514/44 R; 514/44 A; 514/2; 514/34; 528/354

(57) ABSTRACT

This application relates to micelle-forming poly(ethylene oxide)-block-poly(ester) block copolymers having reactive groups on both the poly(ethylene oxide) block and the poly(ester) block therein. The biodegradability of these copolymers and their biocompatibilities with a large number of bioactive agents make them suitable as carriers for various bioactive agents. The bioactive agent, such as DNA, RNA, oligonucleotide, protein, peptide, drug and the like, can be coupled to the reactive groups on the polyester block of the copolymer. A variety of targeting moieties can be coupled to the reactive group on the poly(ethylene oxide) block for targeting the bioactive agent to a particular tissue. The application also relates to a composition and method of use thereof for delivering bioactive agents.

This application claims the benefit under 35 USC 119(e) of U.S. provisional patent application No. 60/870,330, filed Dec. 15, 2006, the disclosure of which is incorporated herein.

### FIELD OF THE INVENTION

The present invention relates to novel ligand guided block copolymers, particularly poly(ethylene oxide)-block-poly(ester) block copolymers having reactive functional groups on the poly(ethylene oxide) (PEO) shell for attaching a variety of targeting moieties. The invention also relates to a composition and method of use thereof for delivering bioactive agents.

### BACKGROUND OF THE INVENTION

Amphiphilic block copolymers can self-assemble to nanoscopic, core/shell structures in which the hydrophobic core acts as a microreservoir for the encapsulation of drugs, proteins or DNA and the hydrophilic shell interfaces the media. Among different block copolymers designed for drug delivery, those with polyethylene oxide (PEO), as the shell-forming block, and polyester or poly amino acids (PLAA), as the core-forming block, are of increasing interest. This is owed to the biocompatibility of PEO and potential biodegradability of polyester and PLAA which make them safe for human administration.

It is generally known that poly amino acids (PLAA) structures are advantageous over polyesters since PLAA can potentially form covalent or electrostatic attachment with drugs, drug compatible moieties, genes or intelligent vectors through free functional groups, such as amine or carboxylic acid, on the amino acid chain. Thus, changes in the length of the hydrophobic/hydrophilic blocks, chemical structure of the side chains and the level of substitution may be used to achieve desired stability, biodegradation, drug loading, release or activation properties.

Through chemical engineering of the core structure in PEO-b-PLAA based micelles, desired properties for the delivery of doxorubicin (DOX), amphotericin B, methotrexate, cisplatin and paclitaxel have been achieved. For instance, a 40 to 50% of DOX substitution and a decrease in the proportion of P(Asp)-DOX to PEO has been used to increase the stability of micelles formed from DOX conjugates of PEO-b-poly(L-aspartic acid). The PEO-b-PAsp-DOX micelles were later utilized to physically encapsulate DOX. Taking advantage of a strong interaction between chemically conjugated and physically encapsulated drug, a novel formulation with efficient solubilization and release properties has been developed for doxorubicin, which is currently in clinical trials in Japan (see Matsumura Y, HamaguchI T, Ura T et al.: Phase I clinical trial and pharmacokinetic evaluation of NK911, a micelle-encapsulated doxorubicin. Br J Cancer (2004) 91(10):1775-1781).

The present inventors have also previously prepared a PEO-b-PLAA based micellar system with saturated fatty acid esters in the core to encapsulate an aliphatic drug, amphotericin B (AmB). The micellar core was fine tuned chemically so that it can effectively sustain the rate of AmB release (see Lavasanifar A, Samuel J, Kwon G S: Micelles of poly(ethylene oxide)-block-poly(N-alkyl stearate L-aspartamide): synthetic analogues of lipoproteins for drug delivery. J Biomed Mater Res (2000) 52(4):831-835). While not wishing to be bound by theory, the formation of more hydrolysable bonds, such as ester bonds, for instance, appears to suggest that micelle-forming block copolymer-drug conjugates with sufficient drug release properties. This approach has been utilized to attach methotrexate (MTX) to PEO-b-PLAA. The level of attached MTX is used to control the stability of the polymeric micelles and the rate of drug release.

While there has been progress made in the design, synthesis and discovery of novel polymeric poly amino acids, the biodegradability of these different structures has not been exploited fully. Although polyesters have had a history of safe application in human, in general, they are less suitable for chemical engineering due to the lack of functional groups on the polymeric backbone. Thus, there remains a need to continually design and develop PEO-b-polyester block copolymers that are biodegradable and biocompatible with a large number of bioactive agents.

It is also desirable to be able to target PEO-b-polyester block copolymers that are biodegradable and biocompatible with bioactive agents to a particular disease site in order to permit lower dosing, reduced side effects, and the like. The present invention is directed towards the preparation of novel poly(ethylene oxide)-block-poly(ester) micelles bearing functional groups on poly(ethylene oxide) (PEO) shell, which may be used to attach a variety of targeting moieties, for example, amine containing ligands (e.g., antibodies, monoclonal antibodies, antibody fragments, sugars, peptides, etc), lipids, oligonucleotides, DNA, RNA, or other small molecules, to the micellar surface and develop a “smart carrier” that can increase the specificity of polymeric micelles for target (diseased) tissue. It may also be desirable to have aromatic, reactive, or conjugated drugs in the micellar core. The presence of functional and aromatic groups in the micellar core provides additional opportunities for fine-tuning of the delivery systems to improve drug encapsulation, enhance micellar stability and control the rate of drug release from the carrier, or chemically attach different drugs, drug compatible moieties or diagnostic agents to the core-forming structure.

### SUMMARY OF THE INVENTION

In one broad aspect, the present application provides poly(ethylene oxide)-block-poly(ester) block copolymers having a reactive functional group on the poly(ethylene oxide) block therein that may be used to attach a variety of targeting moieties useful in targeting the copolymers to a particular target site. In another broad aspect, the present application provides poly(ethylene oxide)-block-poly(ester) block copolymers having at least one, and often multiple, functional/reactive group on the polyester block therein, rendering such copolymers biodegradable and biocompatible with a large number of bioactive agents. Copolymers having at least one functional group on either the poly(ethylene oxide) block or the poly(ester) ester block are herein referred to as “mono-functionalized poly(ethylene oxide)-block-poly(ester) block copolymers”. It is understood that the term “mono-functionalized poly(ethylene oxide)-block-poly(ester) block copolymers” includes those copolymers having more than one functional/reactive groups on the poly(ester) block.

In another broad aspect, the poly(ethylene oxide)-block-poly(ester) block copolymers of the present invention have a reactive functional group on the poly(ethylene oxide) block therein for attaching targeting moieties and at least one, and often multiple, functional/reactive group on the polyester block therein, rendering such copolymers both target-specific and biodegradable and biocompatible with a large number of bioactive agents (herein referred to as “bi-functionalized poly(ethylene oxide)-block-poly(ester) block copolymers”). It is understood that the term “bi-functionalized poly(ethylene oxide)-block-poly(ester) block copolymers” includes those copolymers having more than one functional/reactive groups on the poly(ester) block.

The present application also provides a composition in which either a mono- or bi-functionalized poly(ethylene oxide)-block-poly(ester) block copolymers of the present invention form a micelle around the bioactive agent, thereby forming a shell that is functionalized, a core that is functionalized, or both.

Further, the present application provides a method of use of mono- or bi-functionalized poly(ethylene oxide)-block-poly(ester) block copolymers of the present invention for delivering a bioactive agent to a specific target site.

Accordingly, in one broad aspect, the present application provides a compound of formula I:

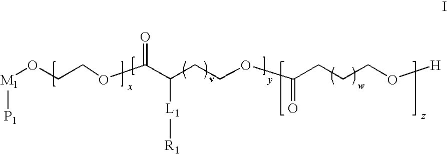

wherein
M₁ is a linker group selected from the group consisting of a single bond, a methyl group, an ethyl group, a propyl group or a C₄₋₁₀alkyl group;
P₁ is CH₃, a reactive functional group or a targeting moiety;
L₁ is a linker group selected from the group consisting of a single bond, —C(O)—O—, —C(O)— and —C(O)NHR²;
R₁ is selected from the group consisting of H, OH, hydrazone, polyamine, polyamine-CF₃, protected polyamine, C₁₋₂₀alkyl, C₃₋₂₀cycloalkyl and aryl, said latter three groups may be optionally substituted and in which one or more of the carbons of the alkyl, cycloalkyl or aryl groups may optionally be replaced with O, S, N, NR² or N(R²)₂ or R₁ is a bioactive agent;
R² is H, NH₂, NH-Fmoc or C₁₋₆alkyl;
v and w are, independently of each other, an integer independently selected from 1 to 4.
x is an integer between 10 and 300;
y is an integer between 5 and 100;
z is an integer between 0 and 100;
wherein aryl is mono- or bicyclic aromatic radical containing from 6 to 14 carbon atoms having a single ring or multiple condensed rings; and
wherein the optional substituents are selected from the group consisting of halo, OH, OC₁₋₆alkyl, C₁₋₆alkyl, C₂₋₆alkenyl, C₂₋₆alkenyloxy, NH₂, NH(C₁₋₆alkyl), N(C₁₋₆alkyl)(C₁₋₆alkyl), CN, NO₂, C(O)C₁₋₆alkyl, C(O)OC₁₋₆alkyl, SO₂C₁₋₆alkyl, SO₂NH₂, SO₂NHC₁₋₆alkyl, phenyl and C₁₋₆alkylenephenyl.

It is understood that the above structure is meant to include both compounds where (1) the functionalized ester(s) of the poly(ester) block are randomly distributed throughout the poly(ester) block and (2) the functionalized ester(s) of the poly(ester) block are present in a block (i.e., together). The process for making both poly(ester) blocks are discussed in more detail below.

In one embodiment, P₁ is a reactive functional group and the reactive functional group is selected from the group consisting of —CH(O—R)₂, where R can be a methyl, ethyl, or any other alkyl group; a carbonyl group; an aldehyde; an alcohol; an amino group; a protected amino group; a carboxyl group; a protected carboxyl group; a mercapto group; a protected mercapto group; a hydrazone; a protected hydrazone; or phenyl or phenyl-alkyl group which has a substituent selected from the group consisting of an acetal group, —C(O—R³)—O—R⁴, a carbonyl group, an aldehyde, an alcohol, an amino group, a carboxyl group and a mercapto group on benzene ring, where R³ and R⁴ are H or C₁₋₆alkyl.

In another embodiment, P₁ is a reactive functional group and the reactive functional group is selected from the group consisting of hydroxyl, protected hydroxyl, active ester, n-hydroxysuccinimidyl, 1-benzotriazolyl, p-nitrophenyl, imidazolyl esters, active carbonate, n-hydroxysuccinimidyl, 1-benzotriazolyl, p-nitrophenyl, imidazolyl carbonate, acetal, aldehyde, aldehyde hydrates, alkyl or aryl sulfonate, halide, disulfide derivatives, o-pyridyl disulfidyl, alkenyl, acrylate, methacrylate, acrylamide, active sulfone, amine, protected amine, hydrazide, protected hydrazide, thiol, protected thiol, carboxylic acid, protected carboxylic acid, isocyanate, isothiocyanate, maleimide, vinylsulfone, dithiopyridine, vinylpyridine, iodoacetamide, epoxide, glyoxals, diones, mesylates, tosylates, or tresylate.

Examples of suitable amino-protecting groups, carboxy-protecting groups and mercapto-protecting groups can be found in U.S. Pat. No. 5,929,177, incorporated herein by reference. For example, if the chemically reactive group of the functional group is an amine, the protecting group can be selected from the group of tert-butyloxycarbonyl (t-Boc) and 9-fluorenylmethoxycarbonyl (Fmoc). If the chemically reactive group is a mercapto group (thiol), the protecting group can be orthopyridyldisulfide. If the chemically reactive group is a carboxylic acid, the protective group can be benzyl or an alkyl group such as methyl, ethyl or tert-butyl.

In one embodiment, M₁ is a bond and P₁ is an alkyl group having 1 to 10 carbon atoms and a terminal carbonyl group. In another embodiment, M₁ is a bond and P₁ is an alkyl group having 2 carbons and a terminal carbonyl group.

In another broad aspect, the present application provides a composition comprising a compound of formula I wherein P₁ is a targeting moiety. As used herein, “targeting moiety” includes any chemical moiety capable of binding to, or otherwise exhibiting an affinity for, a particular type of tissue or component thereof. The addition of a targeting moiety to the copolymer structure can direct the copolymer to particular sites within the body for targeted release of a bioactive agent that is either covalently attached or physically entrapped in a copolymer micelle. Examples of suitable targeting moieties include hydroxyapatite-targeting (bone-targeting) moieties such as bisphosphonates, polyaspartic acid, polyglutamic acid and aminophosphosugars; proteins; antibodies; antibody fragments; peptides; carbohydrates; lipids; oligonucleotides; DNA; RNA; or small molecules having a molecular weight less than 2000 Daltons.

In one embodiment, the targeting moiety is an amine containing ligand selected from the group consisting of monoclonal antibodies, antibody fragments, peptides and carbohydrates. In another embodiment, the targeting moiety is a tumor targeting ligand, preferably a tumor targeting peptide. In another embodiment, the targeting moiety is an integrin ligand, preferably a peptide containing the cell-binding domain -Arg-Gly-Asp-(RGD). In another embodiment, the integrin ligand is Gly-Arg-Gly-Asp-Ser (GRGDS) or Ala-Cys-Asp-Cys-Arg-Gly-Asp-Cys-Phe-Cys-Gly (ACDCRGDCFCG or RGD4C). In another embodiment, the targeting moiety is a neuroblastoma tumor cell-binding peptide, preferably, Val-Pro-Trp-Glu-Pro-Ala-Tyr-Gln-Arg-Phe-Thr (VPWEPAYQRFT or p160).

In another broad aspect, the present application provides a composition comprising a compound of formula I and a bioactive agent, in which the compound of formula I forms a micelle around the bioactive agent. In a more particular embodiment of the invention, the compound of formula I forms a micelle around the bioactive agent by chemical conjugation, electrostatic complexation and physical encapsulation. In another embodiment of the invention, the bioactive agent is selected from the group consisting of DNA, RNA, oligonucleotide, protein, peptide and drug.

In a further embodiment of the invention, the bioactive agent is selected from the group consisting of DNA, other nucleic acid based drugs such as siRNA, oligonucleotides, ribozymes, and the like, protein and a drug. In one embodiment, the drug is selected from the group consisting of cucurbitacins, curcumin, resveratrol, buscopan, celecoxib, doxorubicin (DOX), amphotericin B, methotrexate, cisplatin, paclitaxel, etoposide, cyclosporine A, PSC833, amiodarone, rapamycine, cholesterol and ergoesterol. In a further embodiment of the invention, the drug is selected from doxorubicin (DOX), cholesterol and ergoesterol. In still a further embodiment of the invention, the drug is doxorubicin (DOX). In another embodiment of the invention, the protein is a vaccine.

It is an embodiment of the invention that the optional substituents are selected from the group consisting of halo, OH, OC₁₋₄alkoxy, C₁₋₄alkyl, C₂₋₄alkenyl, C₂₋₄alkenyloxy, NH₂, NH(C₁₋₄alkyl), N(C₁₋₄alkyl)(C₁₋₄alkyl), CN, NO₂, C(O)C₁₋₄alkyl, C(O)OC₁₋₄alkyl, SO₂C₁₋₄alkyl, SO₂NH₂, SO₂NHC₁₋₄alkyl, phenyl and C₁₋₄alkylenephenyl.

In yet another embodiment of the invention, v and w are, independently of each other, 2 or 3.

In yet another embodiment of the invention, v and w are equal. In one embodiment, v and w is 3 (polycaprolactone).

It is an embodiment of the invention that x is an integer between 50 and 200. In a more particular embodiment of the invention, x is an integer between 100 and 150.

In another embodiment of the invention, y is an integer between 5 and 50. In a more particular embodiment of the invention, y is an integer between 10 and 20.

In an embodiment of the invention, z is an integer between 0 and 80, more suitably between 0 and 40.

In accordance with another broad aspect, the block polymers herein may be prepared by a reaction sequence such as the example shown in Scheme A:

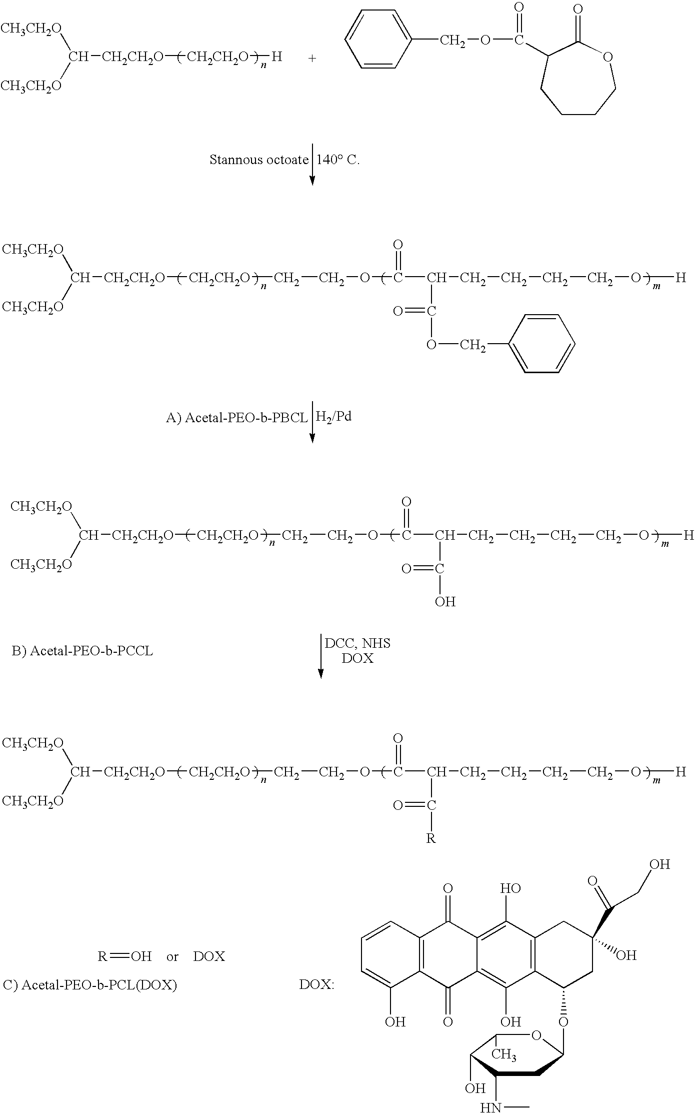

where n is an integer between 10 and 300 and m is an integer between 5 and 100.

In this instance, the functionalized poly(ethylene oxide) block is first reacted with only ester moieties, e.g., caprolactone moieties, that have been functionalized (e.g., α-benzylcarboxylate-ε-caprolactone). Thus, the functionalized caprolactone moieties are present in a block. Thus, these block copolymers will have all of the functionalized esters of the poly(ester) block grouped together in a block.

It is contemplated that the terminal H could be replaced with one or more non-functionalized caprolactone moieties as well. Thus, copolymers can be made where all of the esters are functionalized (e.g., acetal-PEO-b-PBCL and acetal-PEO-b-PCCL) or only some of the esters are functionalized.

In accordance with another broad aspect, the block polymers herein may be prepared by a reaction sequence such as the example shown in Scheme B:

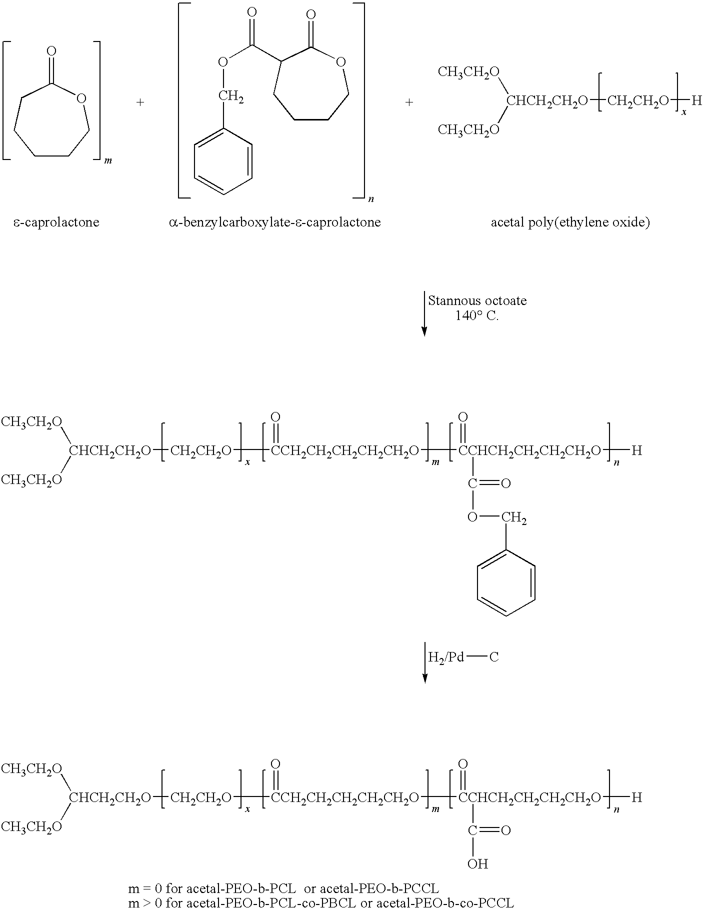

In this instance, the functionalized poly(ethylene oxide) block is reacted with a mixture of both functionalized and non-functionalized ester moieties, e.g., a mixture of caprolactone and α-benzylcarboxylate-ε-caprolactone. Thus, the functionalized caprolactone moieties will be randomly distributed within the poly(ester) block.

In accordance with another broad aspect, the present application provides compounds made according to Scheme A or Scheme B above, for example, acetal-PEO-b-PBCL, acetal-PEO-b-PCCL and acetal-PEO-b-PCL(DOX), which can be further conjugated with a targeting moiety such as an amine containing ligand, for example, a peptide such as GRGDS, RGD4C or p160, according to Scheme C as follows:

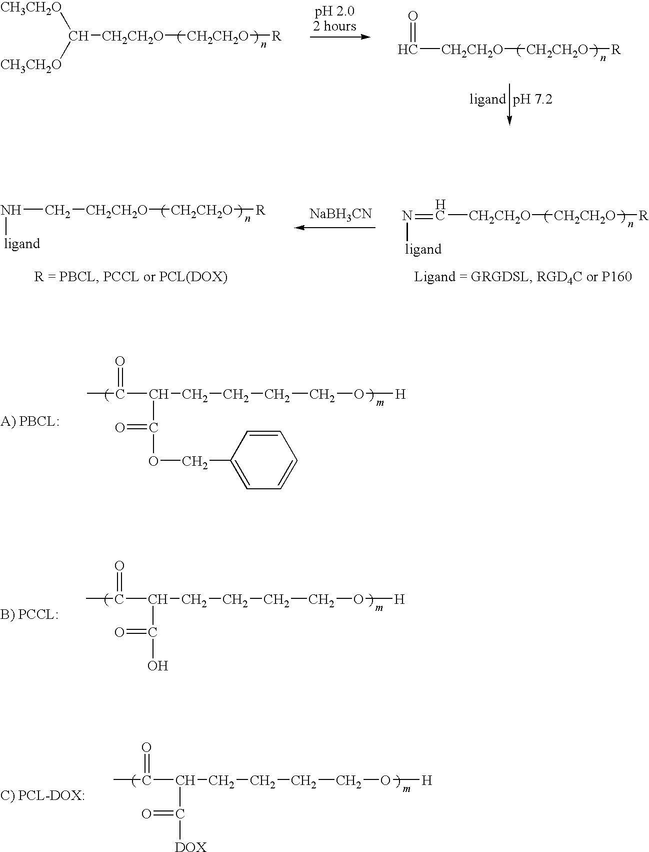

where n is an integer between 10 and 300 and m is an integer between 5 and 100.

Thus, as shown above, the poly(ester) residues of the poly(ethylene oxide)-block-poly(ester) block copolymer of the present invention may be assembled either randomly or in blocks. For example, without being limiting, in a bi-functionalized copolymer where the poly(ester) residues are caprolactone, in the randomly assembled cores both substituted and unsubstituted caprolactone residues are randomly arranged along the length of the core block. With block assembly, a block of substituted caprolactone may be followed by a block of unsubstituted caprolactone (or vice versa). In the alternative, all of the caprolactone residues are substituted.

Also within the scope of the present invention is a method of delivering a bioactive agent to a subject, comprising administering to the subject a compound of formula I which is capable of forming a micelle around an effective amount of the bioactive agent. More particularly, the bioactive agent is selected from the group consisting of DNA, RNA, oligonucleotide, protein, peptide and drug.

For purposes of summarizing the invention and the advantages achieved over the prior art, certain objects and advantages of the invention have been described above. Of course, it is to be understood that not necessarily all such objects or advantages may be achieved in accordance with any particular embodiment of the invention. Thus, for example, those skilled in the art will recognize that the invention may be embodied or carried out in a manner that achieves or optimizes one advantage or group of advantages as taught herein without necessarily achieving other objects or advantages as may be taught or suggested herein.

Other features and advantages of the present invention will become apparent from the following detailed description. It should be understood, however, that the detailed description and the specific examples while indicating preferred embodiments of the invention are given by way of illustration only, since various changes and modifications within the spirit and scope of the invention will become apparent to those skilled in the art from this detailed description.

### BRIEF DESCRIPTION OF THE DRAWINGS

The invention will now be described in relation to the drawings in which:

FIG. 1 shows the ¹H NMR spectrum of α-benzylcarboxylate-ε-caprolactone in CDCl₃ and peak assignments.

FIG. 2 shows the ¹H NMR spectrum of acetal-poly(ethylene oxide) (PEO) in CDCl₃.

FIG. 3 shows the ¹H NMR spectrum of acetal-poly(ethylene oxide)-block-poly(α-benzylcarboxylate-c-caprolactone) (acetyl-PEO-b-PBCL) block copolymer in CDCl₃ and peak assignments.

FIG. 4 shows the ¹H NMR spectrum of acetal-poly(ethylene oxide)-block-poly(α-carboxylate-c-caprolactone) (acetal-PEO-b-PCCL) block co-polymer in CDCl₃ and peak assignments.

FIG. 5 shows the ¹H NMR spectrum of acetal-PEO-b-PCL(DOX) block copolymer in DMSO-d₆ and peak assignments.

FIG. 6(a) shows ¹H NMR spectrum of aldehyde-PEO-b-PBCL block copolymer and peak assignments.

FIG. 6(b) shows ¹H NMR spectrum of aldehyde-PEO-b-PCCL block copolymer in CDCl₃ and peak assignments.

FIG. 6(c) shows ¹H NMR spectrum of aldehyde-PEO-b-PCL(DOX) block copolymer in DMSO-d₆ and peak assignments.

FIG. 7 shows the ¹H NMR spectrum of Phe-PEO-b-PCL block copolymer in DMSO-d₆.

FIG. 8(a) shows the RP-HPLC assessment of aldehyde-PEO-b-PCL+GRGDS.

FIG. 8(b) shows the RP-HPLC assessment of aldehyde-PEO-b-PBCL+GRGDS.

FIG. 8(c) shows the RP-HPLC assessment of aldehyde-PEO-b-PCCL+GRGDS.

FIG. 8(d) shows the RP-HPLC assessment of aldehyde-PEO-b-PCL(DOX)+GRGDS.

FIG. 9(a) shows the RP-HPLC assessment of the peptide conjugation of acetal-PEO-b-PCL+GRGDS.

FIG. 9(b) shows the RP-HPLC assessment of the peptide conjugation of acetal-PEO-b-PBCL+GRGDS.

FIG. 9(c) shows the RP-HPLC assessment of the peptide conjugation of acetal-PEO-b-PCCL+GRGDS.

FIG. 9(d) shows the RP-HPLC assessment of the peptide conjugation of acetal-PEO-b-PCL(DOX)+GRGDS.

FIG. 10(a) shows the RP-HPLC assessment of the peptide conjugation of acetal-PEO-b-PBCL micelles+GRGDS.

FIG. 10(b) shows the RP-HPLC assessment of the peptide conjugation of aldehyde-PEO-b-PBCL micelles+GRGDS.

FIG. 11 shows a plot of % unconjugated GRGDS versus time in hours for acetal-PEO-b-PBCL micelles incubated with GRGDS and aldehyde-PEO-b-PBCL micelles incubated with GRGDS.

FIG. 12 shows the RP-HPLC assessment of the peptide conjugation of aldehyde-PEO-b-PCL(DOX) micelles+RGD4C.

FIG. 13 shows the RP-HPLC assessment of the peptide conjugation of aldehyde-PEO-b-PCL(DOX) micelles+p160.

FIG. 14(a) shows the in vitro cytotoxicity of free DOX, GRGDS-PEO-b-PCL(DOX) and acetal-PEO-b-PCL(DOX) against B16-F10 melanoma cells when cells were incubated with free DOX, GRGDS-PEO-b-PCL(DOX) or acetal-PEO-b-PCL(DOX) for 24 hr.

FIG. 14(b) shows the in vitro cytotoxicity of free DOX, GRGDS-PEO-b-PCL(DOX) and acetal-PEO-b-PCL(DOX) against B16-F10 melanoma cells when cells were incubated with free DOX, GRGDS-PEO-b-PCL(DOX) or acetal-PEO-b-PCL(DOX) for 48 hr.

FIG. 15 shows the ¹H NMR spectrum of α-cholestryl carboxylate-ε-caprolactone (Functionalized monomer).

FIG. 16 shows the IR spectrum of α-cholestryl carboxylate-ε-caprolactone (Functionalized monomer).

FIG. 17 shows the mass spectrum of α-cholestryl carboxylate-ε-caprolactone (Functionalized monomer).

FIG. 18 shows the ¹H NMR spectrum of PEO-b-PChCL block copolymer.

FIG. 19 shows the ¹H NMR spectrum of acetal-PEO-b-P(CL-Hyd-Fmoc) in CDCl₃.

FIG. 20 illustrates the time and pH-dependent DOX release profile of the micelles formed using acetal-PEO-b-P(CL-Hyd-DOX). The micelles selectively released DOX at pH 5.0 but virtually not at all at pH 7.4.

FIG. 21 shows the effect of pH on the fluorescence-quenching effect of the acetal-PEO-b-P(CL-Hyd-DOX) micelles using a fluorescence spectrometer with 485 nm excitation.

FIGS. 22(a) and (b) show the cytotoxicity of cRGDfK-PEO-b-P(CL-Hyd-DOX) micelles with hydrozone as the linker on MDA435/LCC6 sensitive cells and MDA435/LCC6 resistant cells, respectively.

FIG. 22(c) shows the cytotoxicity of RGD4C-PEO-b-P(CL-Hyd-DOX) micelles on MDA435/LCC6 sensitive cells.

FIGS. 23(a) and (b) show the cytotoxicity of RGD4C-PEO-b-P(CL-DOX) micelles with imide as the linker on MDA435/LCC6 sensitive celss and MDA435/LCC6 resistant cells, respectively.

FIG. 24 shows the tumor size changes of the treated SCID mice bearing s.c. MDA435/LCC6 tumors. Treatments were initiated on established 0.1 cm³ tumors, i.e., day 12 after tumor inoculation.

FIG. 25 Survival versus time for SCID mice bearing s.c. MDA435/LCC6 tumors. Treatments were initiated on established 0.1 cm³ tumors, i.e., day 12 after tumor inoculation.

FIGS. 26(a) and (b) is an assessment of P160 conjugation to PEO-PBC1 polymeric micelle using RP-HPLC. The HPLC chromatogram in FIG. 26(a) shows un-reacted free P-160 (200 ug/mL) eluted at 25.5 minutes. The HPLC chromatogram in FIG. 26(b) shows no free P-160 after 72 hrs reaction of aldehyde PEO-b-PBCL micelle with P-160.

FIG. 27 is an assessment of RGDfK conjugation to PEO-PBCL polymeric micelle using RP-HPLC. These two chromatograms show the complete consumption of cRGD by the aldehyde bearing PEO-PBCL due to complete reaction and schiff base formation. HPLC chromatogram of (A) 100 ug/ml free RGDfK showing peak at 13.5 min and (B) aldehyde-PEO-b-PBCL micelle after reaction with RGDfK showing no peaks at 13.5 min.

FIG. 28 is an assessment of cRGD and P160 conjugation to PEO-PBC1 double targeted polymeric micelle using RP-HPLC. HPLC chromatogram of (A) 100 ug/ml free RGDfK showing peak at 13.5 min, (B) 100 ug/ml free P160 showing peak at 25.5 min, (C) aldehyde-PEO-b-PBCL micelle after reaction with RGDfK and P160 peptides showing no peaks at 13.5 or 25.5 min, and (D) acetal-PEO-b-PBCL micelle after reaction with RGDfK and P160 peptides showing peaks at 13.5 and 25.5 min.

FIG. 29(a) shows the cellular uptake of free DiI, DiI loaded P-160 micelles, and DiI unmodified micelles after 3 h of incubation at 37° C. with or without pretreatment with 20 ug/mL of free P-160. Each bar represents average uptake (%)±SD (n=4). NS=non significant; ** P<0.05.

FIG. 29(b) shows the cellular uptake of free DiI, DiI loaded P-160 micelles, and DiI unmodified micelles after 3 h of incubation at 4° C. with or without pretreatment with 20 ug/mL of free P-160. Each bar represents average uptake (%)±SD (n=4). NS=non significant; ** P<0.05.

FIG. 29(c) shows the comparison of cellular uptake of free DiI, DiI loaded P-160 micelles, and DiI unmodified micelles after 3 h of incubation at 37° C. and 4° C. without pretreatment with 20 ug/mL of free P-160. Each bar represents average uptake (%)±SD (n=4). NS=non significant; ** P<0.05.

FIG. 30 shows flow cytometry histograms of (a) DiI loaded P-160 micelles, (b) DiI loaded unmodified micelles and (c) free DiI internalized into MDA-MB-435 cells after 3 hrs incubation at 37° C. (control histogram is cells without any treatment (d)).

### DETAILED DESCRIPTION OF THE INVENTION

### Definitions

The following definitions, unless otherwise stated, apply to all embodiments and aspects of the present invention.

The term “acetal” as used herein means a compound of the formula:

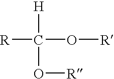

where R is a block polymer of the present invention and R′ and R″ are H or C₁₋₆ alkyl.

The term “aldehyde” as used herein means an organic compound with 1-10 carbon atoms containing a terminal carbonyl group, i.e., a O═CH— group attached to hydrogen or a carbon chain

The term “C₁₋₂₀alkyl” as used herein means straight and/or branched chain alkyl groups containing from one to twenty carbon atoms and includes methyl, ethyl, propyl, isopropyl, t-butyl, pentyl, hexyl and the like.

The term “C₃₋₂₀cycloalkyl” as used herein means saturated cyclic alkyl radicals containing from three to twenty carbon atoms and includes cyclopropyl, cyclobutyl, cyclopentyl, cyclohexyl, cycloheptyl and the like.

The term “aryl” as used herein means a monocyclic or bicyclic carbocyclic ring system containing one or two aromatic rings and from 6 to 14 carbon atoms and includes phenyl, naphthyl, anthraceneyl, 1,2-dihydronaphthyl, 1,2,3,4-tetrahydronaphthyl, fluorenyl, indanyl, indenyl and the like.

The term “C₂₋₆alkenyl” as used herein means straight and/or branched chain alkenyl groups containing from two to six carbon atoms and one to three double bonds and includes vinyl, allyl, 1-butenyl, 2-hexenyl and the like.

The term “C₂₋₆alkenyloxy” as used herein means straight and/or branched chain alkenyloxy groups containing from two to six carbon atoms and one to three double bonds and includes vinyloxy, allyloxy, propenyloxyl, butenyloxy, hexenyloxy and the like.

The term “alkylene” as used herein means bifunctional straight and/or branched alkyl radicals containing the specified number of carbon atoms.

The term “bioactive agent” as used herein means any biologically active moiety which can affect any physical or biochemical properties of a biological organism and includes any substance intended for diagnosis, cure mitigation, treatment, or prevention of diseases in humans and other animals, or otherwise enhance physical or mental well-being of humans or animals.

The term “halo” as used herein means halogen and includes chloro, fluoro, bromo, iodo and the like.

The term “an effective amount” of an agent as used herein is that amount sufficient to effect beneficial or desired results, including clinical results, and, as such, an “effective amount” depends upon the context in which it is being applied. For example, in the context of administering an agent that acts as a drug, an effective amount of an agent is, for example, an amount sufficient to achieve a therapeutic response as compared to the response obtained without administration of the agent.

The term “subject” as used herein includes all members of the animal kingdom including human. The subject is preferably a human.

The term “biodegradable” as used herein means the conversion of materials into less complex intermediates or end products by solubililization hydrolysis, or by the action of biologically formed entities which can be enzymes and other products of the organism.

The term “biocompatible” as used herein means materials or the intermediates or end products of materials formed by solubilization hydrolysis, or by the action of biologically formed entities which can be enzymes and other products of the organism and which cause no adverse effects to the body.

The term “critical micelle concentration” as used herein means the concentration of copolymers above which micelles are spontaneously formed.

### General Description

A number of polyesters have been explored as the core material for copolymer micelle drug delivery systems. It was discovered, however, that esters such as caprolactone have inherent properties that make them suitable to form highly stable micelles. For example, compared to other polyesters used for micelle-forming block copolymers, caprolactone has a longer CH₂ chain and is therefore more hydrophobic. This hydrophobicity gives PEO-PCL micelles a lower critical micelle concentration (CMC), resulting in micelles with greater thermodynamic stability. In a therapeutic setting, this means that such copolymers remain stable in micellar form even when diluted in blood circulation.

Further, PCL is a semi-crystalline polymer in its solid state. This property combined with hydrophobic interactions between PCL chains create kinetic stability in PEO-PCL micelles. A kinetically stable micelle takes longer to dissociate into unimers when diluted below CMC, allowing even thermodynamically unstable micelles to avoid dissociation. (Liu, Zeng and Allen, Eur. J. Pharm & Biopharm (2007) 65 309-319). Thus PEO-PCL micelles have advantageous properties for drug delivery.

However, the inability to add functional groups along the length of the PCL core block has previously confined the use of PEO-b-PCL to physical encapsulation of highly hydrophobic drugs. The present application describes the addition of multiple side functional groups to the caprolactone core block of PEO-b-PCL. Biodegradable micelle-forming poly(ethylene oxide)-block-poly(ester) (e.g., PEO-b-PCL) block copolymers with functional groups on the poly(ester) block (e.g., PCL block) were prepared for incorporating bioactive agents. It was found that the introduction of functional groups, for example, at least five functional groups, to the polyester segment of PEO-b-polyester block copolymers such as PEO-b-poly(ε-caprolactone) (PEO-b-PCL) resulted in the development of biodegradable, self-assembling biomaterials with a potential for the attachment of different reactive compounds to the core-forming structure.

Functional groups permit therapeutics or linker groups to be conjugated directly to the micellar core, expanding the nature of the drugs that can be delivered with PEO-b-PCL and providing a means to optimize micellar drug delivery characteristics. Changing the nature and number of the copolymer's functional side groups can provide tailored loading and release for many different molecules.

The addition of functional groups to the PCL block gives the added advantage of increased micellar thermodynamic and kinetic stability, as seen in the lower CMC and lower Ie/Im ratio of the PEO-PBCL and PEO-PCCL copolymers compared to those for PEO-PCL shown in Table 2. Further, the incorporation of hydrolyzable linker groups on the PCL core allows pH triggered release of the micelle's drug contents in an acidic environment, e.g. in the acidic microenvironment of solid tumors or within intracellular organelles, leading to a better release and cytotoxicity profile for the conjugated drug. The drug remains stable within the micelle while in circulation, and is released preferentially at its site of action.

Specifically in the example of PEO-P(CL-Dox), conjugation of doxorubicin to the block copolymer and further self association of the copolymers to micellar structure is expected to minimize the chance of DOX leakage from carrier during blood circulation and restrict the distribution of conjugated drug only to tissues accessible for the carrier. As a result, the conjugated DOX will follow the fate of the polymeric micellar delivery system, circulate for longer period in blood and preferentially accumulate in solid tumor by enhanced permeability and retention (EPR) effect (Maeda, Adv Drug Del Rev (2001)169-185; Muggia, Clin Canc Res (1999) 5: 7-8).

Thus, PEO-b-P(CL-DOX) conjugates herein may provide one or more of the following benefits: a) a possibility for the incorporation of several DOX molecules per polymer chain, which can lower the required polymer dose of administration; b) thermodynamic stability induced by a great tendency for micellization due to the presence of hydrophobic PCL backbone as the core forming block; c) stabilization of DOX within the carrier through covalent conjugation to the polymer and further self association of the polymer, which will lower the chance of premature DOX release in blood circulation; or d) degradability of the hydrophobic backbone, to which DOX is covalently attached, by hydrolysis which is catalyzed in acidic conditions found within the tumor microenvironment and within cellular organelles.

Further, poly(ethylene oxide)-block-poly(ester) micelles bearing reactive functional groups on poly(ethylene oxide) (PEO) shell together with the aromatic groups, reactive groups, or conjugated drugs in the micellar core have been prepared (bi-functionalized copolymers). The functional group on micellar shell may be used to attach a variety of targeting moieties, for example, monoclonal antibodies, antibodies fragments, sugars, peptides, and the like, to the micellar surface for targeting diseased tissue. Thus, the presence of functional groups on both the micellar shell and the micellar core provides a delivery system that results in one or more of the following unexpected properties: improved drug encapsulation, enhanced micellar stability, controlled rate of drug release from the carrier, and targeted drug delivery to the tissue of interest.

The present invention also includes a method of delivering a bioactive agent to a subject, comprising administering to the subject a compound of formula I as defined above which is capable of forming a micelle around an effective amount of the bioactive agent. More particularly, the bioactive agent is selected from the group consisting of DNA, RNA, oligonucleotide, protein, peptide and drug.

One example of a bi-functional biodegradable amphiphilic copolymer of the present application that was synthesized is acetal-PEO-b-PBCL. The anticancer drug, doxorubicin (DOX) was conjugated to the side chain of polyester section by amide bond or pH-sensitive bond. After preparation of micelles, various specific cancer-targeted peptides (i.e., cRGDfK, RGD4C and P160 etc.) were installed on the micellar surface for active tumor-targeting. The results presented herein show that modification of the PEO shell by integrin specific ligands (i.e., RGD containing peptide) resulted in enhanced micellar uptake by integrin overexpressing cancer cells via integrin-mediated endocytosis, which led to an increased tumor cell-killing activity for both encapsulated and conjugated DOX in their corresponding polymeric micellar carriers. The facilitated uptake of GRGDS-modified micelles exposes the micellar carrier containing either physically encapsulated or chemically conjugated DOX to the acidic and harsh environment of the endosomes/lysosomes necessary for PCL backbone hydrolysis and micellar dissociation. As a result, the release of physically encapsulated DOX from its polymeric micellar carrier is expected to be greatly augmented. Premature DOX release from micelles upon dilution in blood after i.v. injection is expected to be avoided to great extent by the polymeric micellar DOX conjugate, leading to higher DOX accumulation in tumor by the polymeric micellar drug conjugate compared to DOX physically encapsulated in polymeric micelles. The higher tumor accumulation of DOX in vivo may compensate for the decreased cytotoxicity of DOX caused by its conjugation to polymer observed in in vitro studies. Simultaneous modification of the micellar shell and core will modulate the performance of the incorporated drug and points to the potential of the multi-functionalized PEO-b-poly(ester)s for the development of customized nano-delivery systems.

In another example, peptide-modified PEO-b-PCL micelles with conjugated DOX by pH sensitive hydrozone bond (peptide-PEO-b-P(CL-Hyd-DOX) was synthesized. Cellular uptake of P160-modified PEO-b-PBCL micelles by MDA435/LCC6 sensitive cells was evaluated. The cytotoxicity of RGD4C-modified PEO-b-P(CL-DOX) on MDA435/LCC6 sensitive or resistant cells and the cytotoxicity of RGDfK or RGD4C-modified PEO-b-P(CL-Hyd-DOX) on MDA435/LCC6 sensitive or resistant cells were determined. Finally, the therapeutic efficacy of RGD4C-modified PEO-b-P(CL-Hyd-DOX) on SCID mice bearing MDA435/LCC6 sensitive tumors was illustrated. The results presented herein show that these micelles are stable over a long period of time and that more of the drug is released at pH 5.0 than at ph 7.4. eg. in the acidic microenvironment of solid tumors or within intracellular organelles, leading to a better release and cytotoxicity profile for the conjugated drug.

The following non-limiting examples are illustrative of the invention:

### Experimental Examples

While the present invention has been described with reference to what are presently considered to be the preferred examples, it is to be understood that the invention is not limited to the disclosed examples. To the contrary, the invention is intended to cover various modifications and equivalent arrangements included within the spirit and scope of the appended claims.

All publications, patents and patent applications are herein incorporated by reference in their entirety to the same extent as if each individual publication, patent or patent application was specifically and individually indicated to be incorporated by reference in its entirety. Where a term in the present application is found to be defined differently in a document incorporated herein by reference, the definition provided herein is to serve as the definition for the term.

### Materials:

Diisopropyl amine (99%) Benzyl chloroformate (tech. 95%), Sodium (in Kerosin) and butyl lithium (Bu-Li) in hexane (2.5 M Solution) were purchased from Sigma chemicals (St. Louis, Mo., USA). ε-caprolactone was purchased from Lancaster Synthesis, UK. Stannous octoate was purchased from MP biomedicals Inc, Germany. Ethylene oxide (EO) was distilled twice, firstly in the presence of potassium hydroxide and secondly in the presence of calcium hydride. 3,3-Diethoxy-1-propanol (DEP), naphthalene and potassium, DCC and NHS were bought from Sigma-Aldrich (St. Louis, Mo.) and used as received. GRGDS was purchased from Bachem (Torrense, Calif.). Potassium naphthalene solution was prepared by conventional method [34] and the concentration was determined by titration. All other chemicals were reagent grade. Cell culture media RPMI 1640, penicillin-streptomycin, fetal bovine serum, L-glutamine and HEPES buffer solution (1 M) were purchased form GIBCO, Invitrogen Corp (USA). Doxorubicin was purchased from Hisun Pharmaceutical Co. (Zhejiang, China).

### Example 1

### Synthesis of α-benzylcarboxylate-ε-caprolactone

The scheme for synthesis of α-benzylcarboxylate-ε-caprolactone is shown in Scheme 1 below.

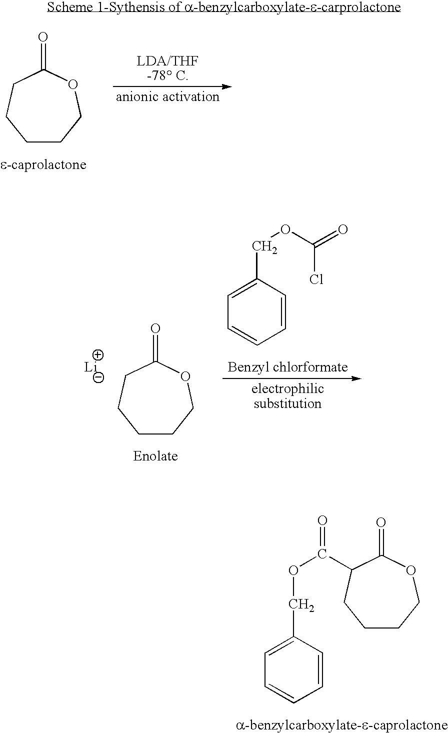

To a solution of 60.0 mmol (8.4 mL) of dry diisopropylamine in 60 mL of dry THF in a 3 neck round bottomed flask, 60.0 mmol (24 mL) of BuLi in hexane was added slowly at −30° C. under vigorous stirring with continuous argon supply. The solution was cooled to −78° C. and kept stirring for additional 20 minutes. Freshly distilled ε-caprolactone (30 mmol or 3.42 g) was dissolved in 8 mL of dry THF and added to the above mentioned mixture slowly, followed by the addition of benzyl chloroformate (30 mmol, 5.1 g) after 45 minutes. The temperature was allowed to rise to 0° C. after 1.5 h and the reaction was quenched with 5 mL of saturated ammonium chloride solution. The reaction mixture was diluted with water and extracted with ethyl acetate (3×40 mL). The combined extracts were dried over Na₂SO₄ and evaporated. The yellowish oily crude mixture was purified over a silica gel column using hexane:ethyl acetate 3:1, 2:1 and 1:1 ratios as eluent. Yield of the reaction (%) was calculated by the following equation:

$$yield ( \% ) = \frac{The actuall amount of the reaction product}{The predicted amount of the reaction product} \times 100$$

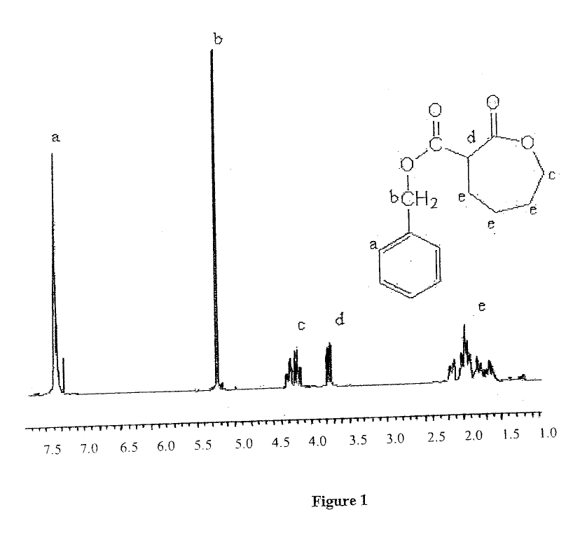

After column chromatography α-benzylcarboxylate-ε-caprolactone was isolated as clear thick oily liquid. The yield of reaction was 53.8%. ¹H NMR (CDCl₃) at 300 MHz: δ=1.6-2.2 (m, 6H); δ: 3.75 (d, 1H); δ: 4.13-4.35 (m, 2H); δ: 5.226 (s, 2H); δ: 7.4 (s, 5H) (FIG. 1).

### Example 2

### Synthesis of acetal poly(ethylene oxide)

Acetal-PEO was synthesized by anionic ring-opening polymerization at room temperature under argon stream adopting a previously reported method for the preparation of acetal-PEO-b-PDLLA with some modifications as shown in Scheme 2 below.

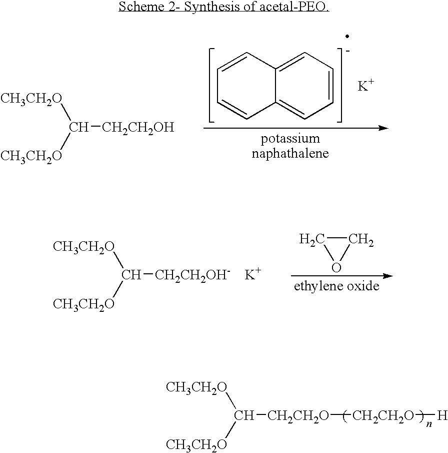

Briefly, 1 mmol (0.15 mL) of initiator (3,3-diethoxy-1-propanol) and 1 mmol (3.5 mL) potassium naphthalene were added to 20 mL of dry THF. After 10 min of vigorous stirring, 114 mmol (5.7 mL) of condensed ethylene oxide (EO) was added via a cooled syringe to the mixture. The polymerization of EO proceeded for 2 days at room temperature under argon resulting in a highly viscous solution. A part of the reaction product was sampled to follow the progression of EO polymerization by GPC. The number average molecular weight of PEO was estimated from its ¹H NMR spectrum comparing the peak intensity ratio of the methine proton in acetal residue ((CH₃CH₂O)₂CH—, δ=4.6) and the methylene protons (—CH₂CH₂O—, δ=3.65) of PEO.

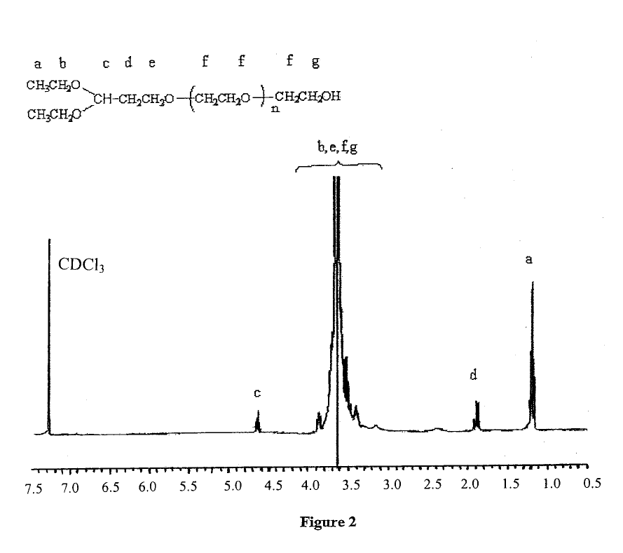

The yield for the preparation of acetal-PEO was 80%. ¹H NMR (CDCl₃) at 300 MHz: δ: 1.20 (tri, 6H); δ: 1.95 (quartet, 2H); δ: 3.65 (s, 4H); δ: 4.10 (tri, 2H) (FIG. 2). The molecular weight of prepared acetal-PEO measured by ¹H NMR was 3600 g·mol⁻¹ (Table 1).

### Example 3

### Synthesis of acetal polyethylene oxide)-block-poly(α-benzylcarboxylate-ε-caprolactone) (acetal-PEO-b-PBCL) block copolymer

Acetal-PEO-b-PBCL was synthesized by ring opening polymerization of α-benzylcarboxylate-ε-caprolactone using acetal-poly(ethylene oxide) as initiator and stannous octoate as catalyst. Synthetic scheme for the preparation of the block copolymer is shown in Scheme 3 below.

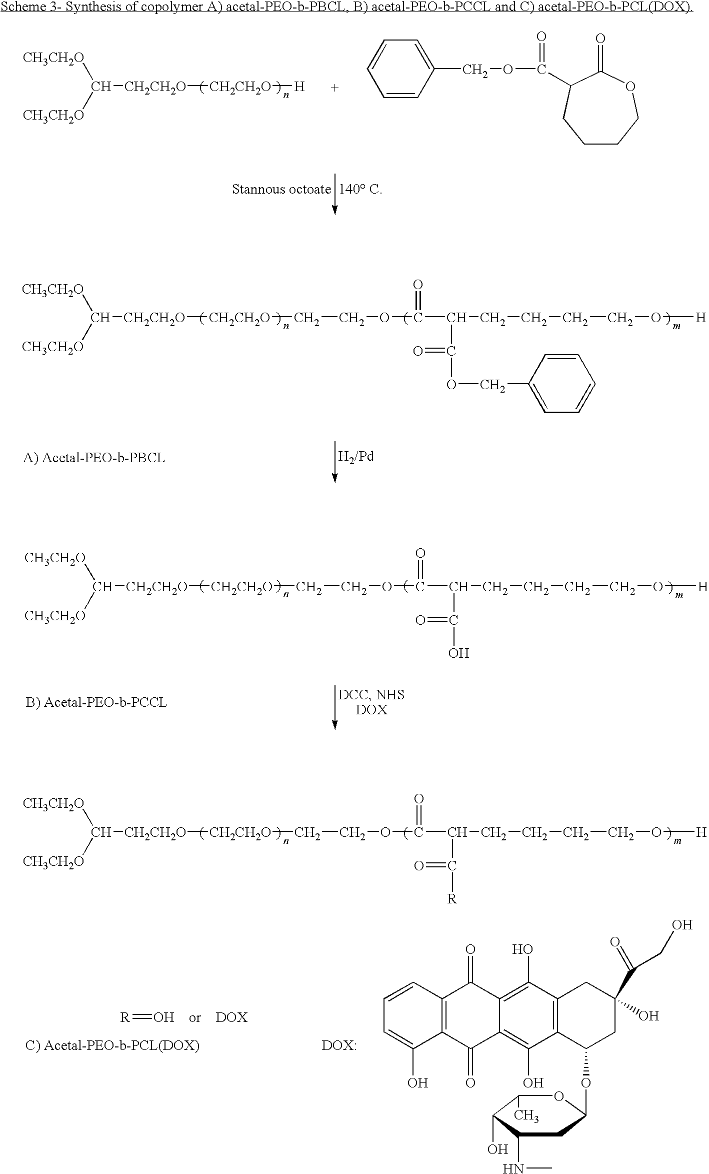

Acetal-PEO (MW: 3500 gm/mole) (3.5 g), α-benzylcarboxylate-ε-caprolactone (3.5 g) and stannous octoate (0.002 eq of monomer) were added to a 10 mL previously flamed ampoule, nitrogen purged and sealed under vacuum. The polymerization reaction was allowed to proceed for 4 hs at 140° C. in oven. The reaction was terminated by cooling the product to room temperature.

¹H NMR spectrum of acetal-PEO-b-PBCL in CDCl₃ at 300 MHz was used to assess the conversion of α-benzylcarboxylate-ε-caprolactone monomer to PBCL comparing peak intensity of —O—CH2- (δ=4.25) for α-benzylcarboxylate-ε-caprolactone monomer to the intensity of the same peak for PBCL (δ=4.05). The number average molecular weight of the block copolymers was also determined from ¹H NMR spectrum comparing peak intensity of PEO (—CH₂CH₂O—, δ=3.65) to that of PBCL (—O—CH₂—, δ=4.05).

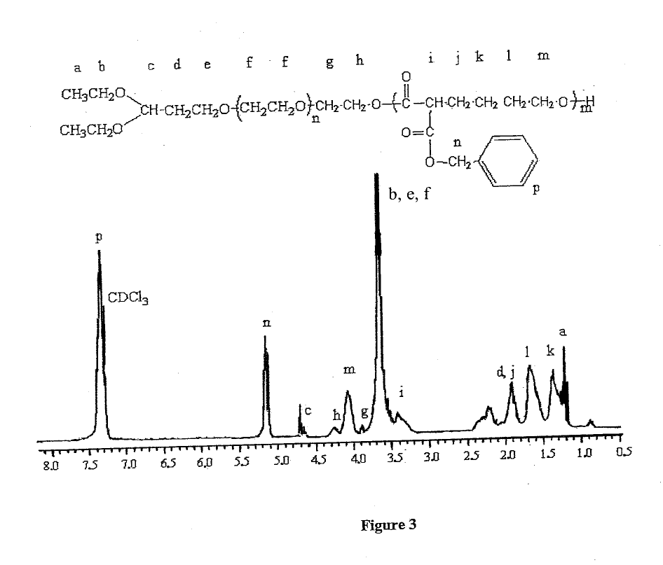

The yield for the preparation of block copolymer was 90%. ¹H NMR (CDCl₃) at 300 MHz: δ: 1.20 (tri, 6H); δ: 1.25-2.0 (m, 6H); δ: 3.65 (s, 4H); δ: 4.05 (tri, 2H); δ: 4.65 (tri, 1H); δ: 5.15 (s, 2H); δ: 7.35 (s, 5H) (FIG. 3). The molecular weight of prepared acetal-PEO-b-PBCL block copolymer measured by comparing the peak intensity of PEO to that of PBCL in the ¹H NMR spectrum was calculated to be 6660 g·mol⁻¹ (Table 1).

### Example 4

### Synthesis of acetal polyethylene oxide)-block-poly((α-carboxylic-ε-caprolactone) (acetal-PEO-b-PCCL) block copolymer

Carboxyl group bearing block copolymers, i.e., acetal-PEO-b-PCCL was obtained by the catalytic debenzylation of acetal-PEO-b-PBCL in the presence of hydrogen gas (Scheme 3). Briefly, a solution of acetal-PEO-b-PBCL (1 g in 25 mL of THF) was placed into a 100 mL round bottom flask. Charcoal coated with palladium (300 mg) was dispersed in this solution. Vacuum was applied for 10 minutes and hydrogen gas was introduced to the reaction flask. The mixture was stirred vigorously with a magnetic stirrer and reacted with hydrogen for 24 h. The reaction mixture was centrifuged at 3000 rpm to remove the catalyst. The supernatant was collected, condensed under reduced pressure and precipitated in diethyl ether and washed repeatedly to remove impurities. The final product was collected and dried under vacuum at room temperature for 48 h. ¹H NMR spectrum of reduced block copolymer in CDCl₃ at 300 MHz was used to assess the conversion of α-benzyl carboxylate to carboxyl group following the disappearance of the characteristic aromatic peak at δ=7.4 ppm.

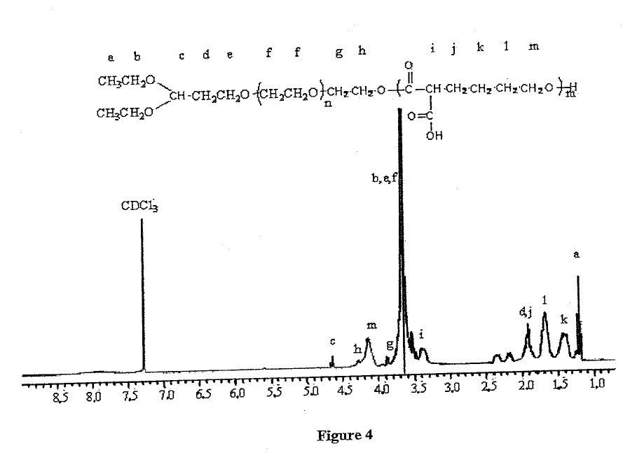

The yield for the preparation of block copolymer was 80%. ¹H NMR (CDCl₃) at 300 MHz: δ: 1.20 (tri, 6H); δ: 1.25-2.0 (m, 6H); δ: 3.65 (s, 4H); δ: 4.10 (tri, 2H); δ: 4.65 (tri, 1H) (FIG. 4). The molecular weight of prepared acetal-PEO-b-PCCL block copolymer measured by comparing the peak intensity of PEO to that of PCCL in the ¹H NMR spectrum was calculated to be 5070 g·mol⁻¹(Table 1).

### Example 5

### Synthesis of α-cholesteryl carboxylate-ε-caprolactone

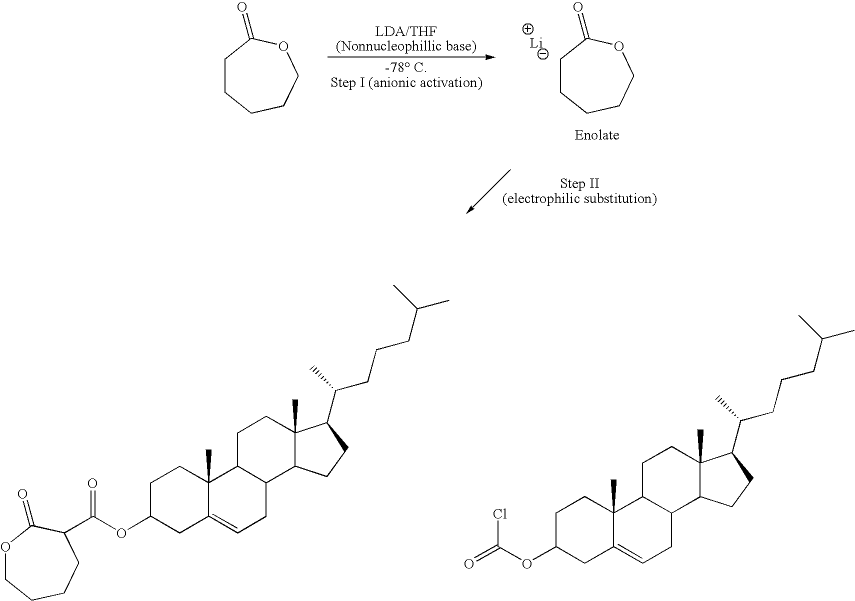

A 60.0 mmol (24 mL) solution of BuLi in hexane was slowly added to a solution of 60.0 mmol (8.4 mL) of dry diisopropylamine in 45 mL of dry THF in a 3 neck round bottomed flask at −30° C. under vigorous stirring with continuous argon supply. The solution was cooled to −78° C. and kept stirring for additional 20 minutes. Freshly distilled ε-caprolactone (30 mmol or 3.42 g) was dissolved in 8 mL of dry THF and added to the above mentioned mixture slowly, followed by the addition of cholesteryl chloroformate (30 mmol, 13.47 g) after 45 minutes. The temperature was raised to 0° C. after 1.5 h and the reaction was quenched with 5 ml of saturated ammonium chloride solution. The reaction mixture was diluted with water and extracted with ethyl acetate (3×40 ml). The combined extracts were dried over Na₂SO₄ and evaporated. The yellowish solid crude mixture was purified over a silica gel column using hexane:ethyl acetate 3:1 ratio as eluent to get solid white powder. The collected fraction was again purified with solvent-solvent extraction using chloroform; hexane and chloroform; methanol solvent system to get the pure solid white powder.

After column chromatography α-cholesteryl carboxylate-ε-caprolactone was isolated as white solid powder. The yield of reaction was around 50%. The structure was confirmed by combined analysis of ¹H NMR, IR and Mass spectroscopy.

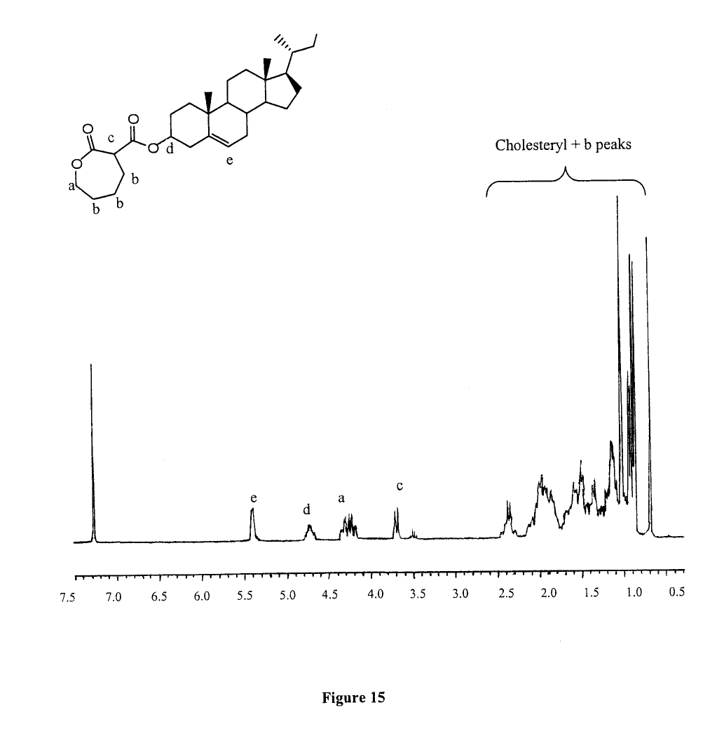

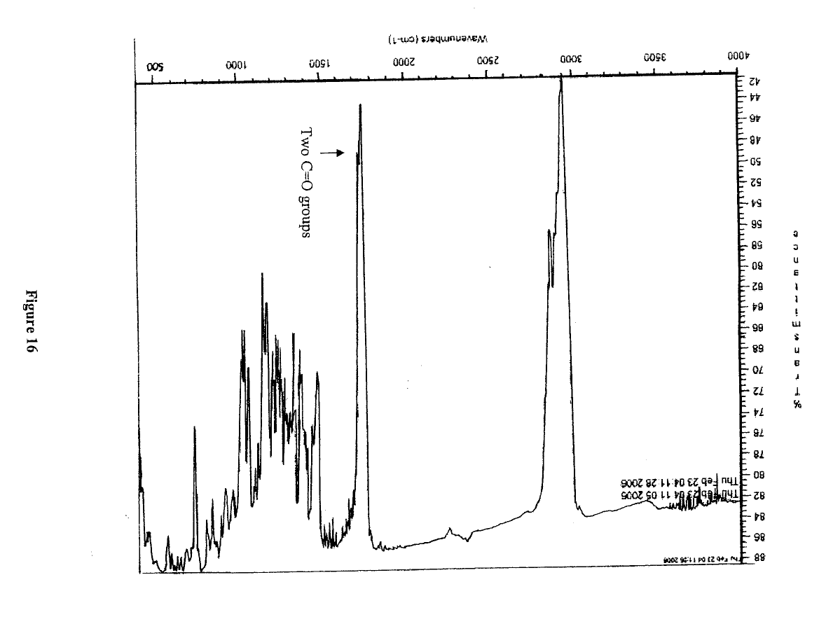

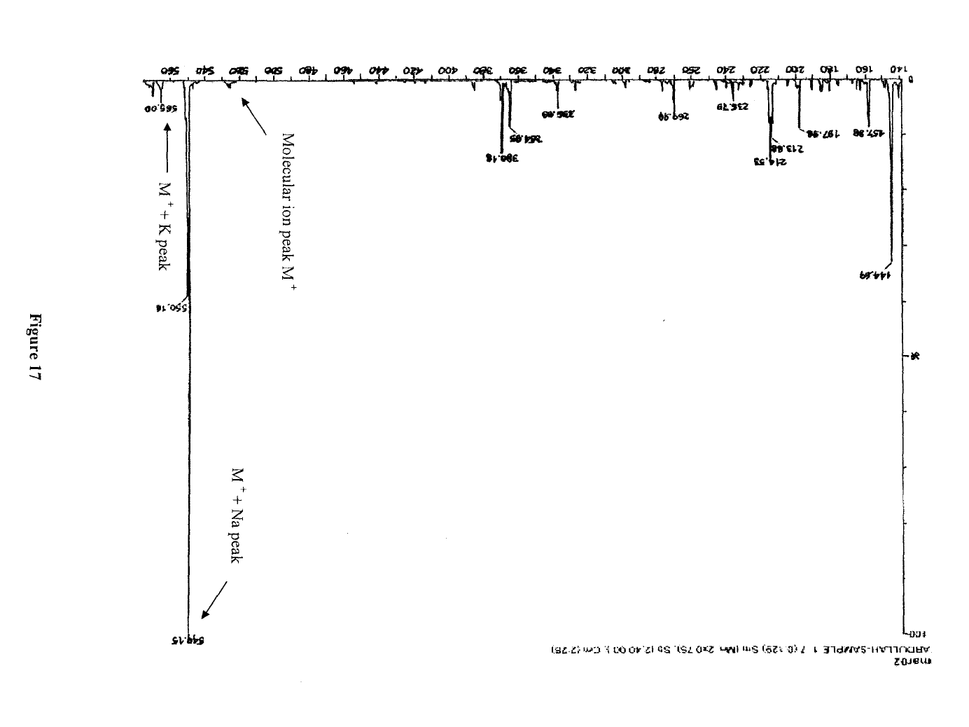

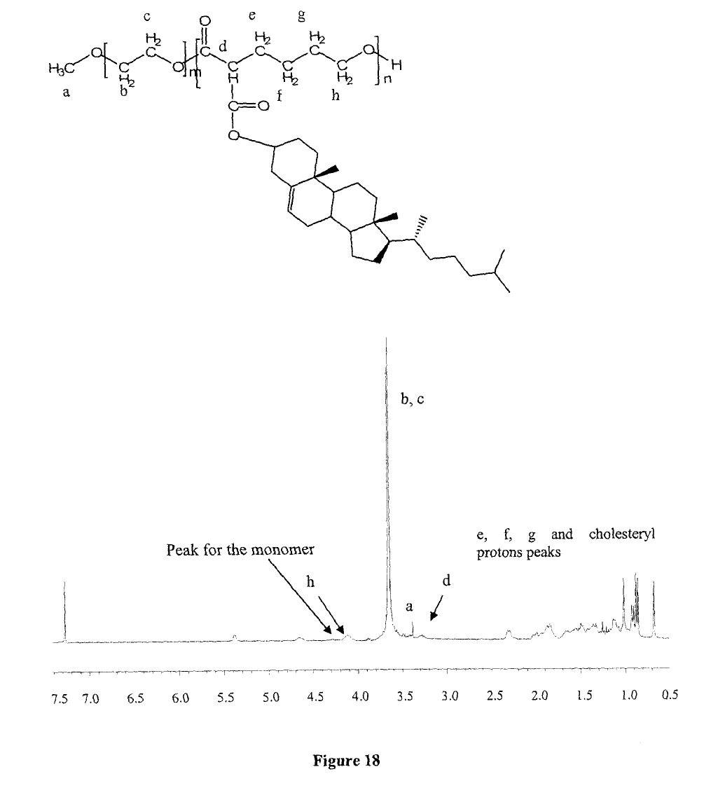

¹H NMR (CDCl₃) at 300 MHz: δ=0.681 (s, 3H) δ: 0.86-1.7 (m, 36H); δ: 1.8-2.1 (m, 12H); δ: 2.35 (m, 2H); δ:3.66 (dd, 1H), δ: 4.13-4.35 (m, 2H); δ: 4.7 (m, 1H) δ: 5.38 (s, 2H) (FIG. 15).

IR spectrum (FIG. 16) shows two adjacent bands at 1725 cm⁻¹ and 1750 cm⁻¹ that indicate the presence of two carbonyl group compared to the IR spectrum of cholesteryl chloroformate (not shown) that shows only one sharp band at 1775 cm⁻¹.

Mass analysis: Peaks: M⁺ m/z: 526.76; M⁺+Na: m/z: 549.15; M⁺+K=m/z: 565.09 (FIG. 17).

### Example 6

### Synthesis and characterization of poly(ethylene oxide)-block-poly(α-cholesteryl carboxylate-ε-caprolactone) (PEO-b-PChCL) block copolymer

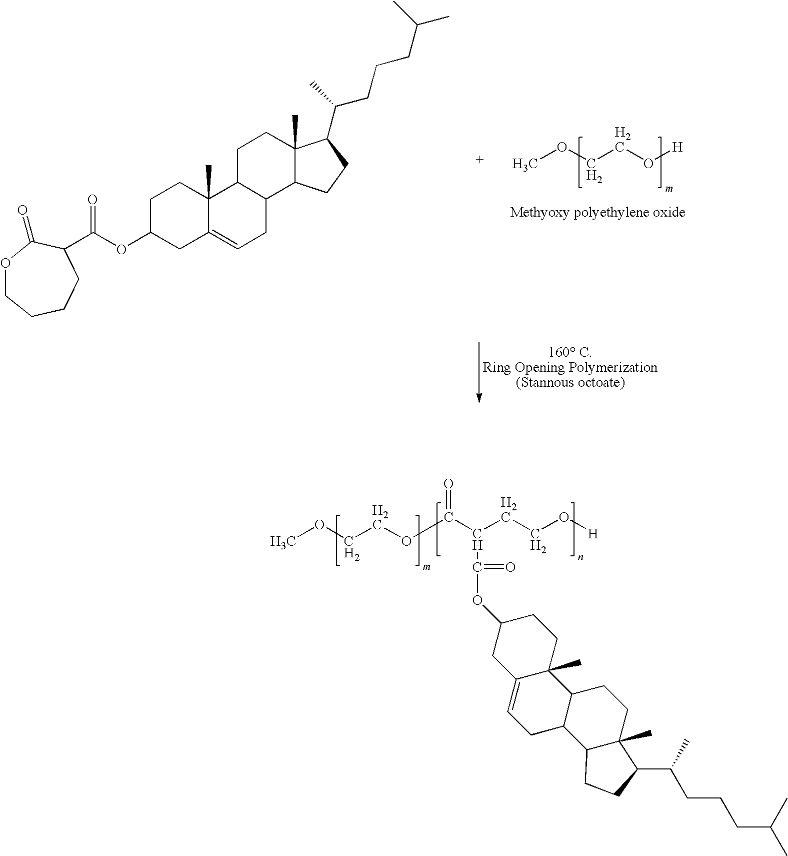

PEO-b-PChCL was synthesized by ring opening polymerization of α-cholesteryl carboxylate-ε-caprolactone using methoxy polyethylene oxide as initiator and stannous octoate as catalyst. Methoxy PEO (MW: 5000 gm/mole) (3.5 g), α-cholesteryl carboxylate-ε-caprolactone (3.5 g) and stannous octoate (0.002 eq of monomer) were added to a 10 mL previously flamed PEO-b-PChCL block copolymer purged and sealed under vacuum. The polymerization reaction was allowed to proceed for 3 h at 160° C. in oven. The reaction was terminated by cooling the product to room temperature.

¹H NMR spectrum of PEO-b-PChCL in CDCL₃ at 300 MHz was used to assess the conversion of α-cholesteryl carboxylate-ε-caprolactone monomer to PChCL comparing peak intensity of —O—CH₂— (δ=4.28 ppm) for α-cholesteryl carboxylate-ε-caprolactone monomer to the intensity of the same peak for PChCL (δ=4.10 ppm). The number average molecular weight of the block copolymers was also determined from ¹H NMR spectrum comparing peak intensity of PEO (—CH₂CH₂O—, δ=3.65 ppm) to that of PChCL (—O—CH₂—, δ=4.10 ppm) (FIG. 18).

The yield for the preparation of PEO-b-PChCL block copolymer was 50%. ¹H NMR (CDCl₃) at 300 Mhz: δ=δ=0.681 (s, 3H) δ: 0.86-1.7 (m, 36H); δ: 1.8-2.1 (m, 12H); δ: 2.3 (m, 2H); δ: 3.28 (tri, 1H), δ: 4.10 (m, 2H); δ: 4.65 (m, 1H) δ: 5.38 (s, 2H) (FIG. 16). The molecular weight of prepared PEO-b-PChCL block copolymer measured by comparing the peak intensity of PEO to that of PBCL in the ¹H NMR spectrum was calculated to be 7633 g·mol⁻¹. ¹H NMR spectrum of PEO-b-PChCL block copolymer (FIG. 16) shows a shift of the protons belong to c-caprolactone ring to upfield compared to the ¹H NMR of monomer (FIG. 15) i.e, Peaks at δ: 4.28 (m, 2H) for O—CH₂ shifts to δ: 4.10; peak at δ: 3.66 (dd, 1H) for O═C—CH— shifts to 3.28 ppm. These shifts indicate the ring opening polymerization of α-cholesteryl carboxylate-ε-caprolactone to form PEO-b-PChCL block copolymer.

### Example 7

### Synthesis of doxorubicin-conjugated acetal polyethylene oxide)-block-poly(caprolactone) (acetal-PEO-b-PCL-DOX) block copolymer

Doxorubicin was conjugated to the acetal-PEO-b-PCL by amide bond formation between the amino group of DOX and the free carboxyl groups on the PCCL chain (Scheme 3). Briefly, to a solution of acetal-PEO-b-PCCL (50 mg, ˜0.01 mmol) in 10 mL of dry THF, 1,3-dicyclohexycarbodiimide (DCC) and N-hydroxy succimide (NHS) in tetrahydrofuran was added. The reaction was allowed for 5 h till precipitate was formed. Then, DOX (2 mg, 0.0036 mmol) was dissolved in THF and 1.3 equivalents of triethylamine was added drop-wise to the polymer solution. The reaction proceeded for another 24 h in room temperature. The resulting solution was centrifuged to remove the precipitate followed by evaporation under vacuum to remove the solvents. 10 mL of methanol was then introduced to dissolve the product. The resulting solution was first purified by Sephadex LH column using methanol as the eluent, and then dialyzed (molecular weight cut off of 3500 Da) extensively against water to remove free DOX and freeze-dried for further use. The content of conjugated DOX was determined by measuring its absorbance at 485 nm, on the assumption that molar absorptivity of DOX residue bound to the polymer was identical to that of free DOX at 485 nm. DOX content was expressed in mol % with respect to the α-carboxylic-c-caprolactone residue of acetal-PEO-b-PCCL, which was estimated from the peak intensity ratio of the aromatic protons (δ=7.35 ppm) of DOX to that of methene (—CH₂O—, δ=4.05 ppm) in the PCL segment of the block copolymer.

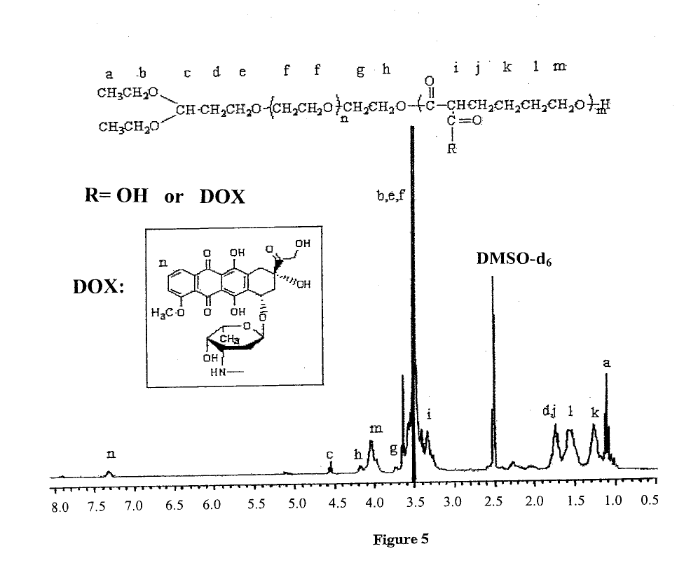

The yield for the preparation of block copolymer was 73%. ¹H NMR (DMSO-d₆) at 300 MHz: δ: 1.20 (tri, 6H); δ: 1.25-2.0 (m, 6H); δ: 3.65 (s, 4H); δ: 4.05 (tri, 2H); δ: 4.55 (tri, 1H); δ: 7.35 (s, 3H) (FIG. 5). The molecular weight of prepared acetal-PEO-b-PBCL block copolymer measured by comparing the peak intensity of PEO to that of PBCL in the ¹H NMR spectrum was calculated to be 5450 g·mol⁻¹ (Table 1). The degree of DOX conjugation to the block copolymer was 9.1 mol %.

### Example 8

### Assembly of Block Copolymers and Characterization of Self-Assembled Structures

Micellization was achieved by dissolving each block copolymer (20 mg) in acetone (0.8 mL) and drop-wise addition (˜1 drop/15 sec) of polymer solution to doubly distilled water (5 mL) under moderate stirring at 25° C., followed by evaporation of acetone under vacuum. Average diameter and size distribution of prepared micelles were estimated by dynamic light scattering (DLS) using Malvern Zetasizer 3000 at a polymer concentration of 4 mg/mL.

### Example 9

### Conversion of acetal group on the acetal-PEO-b-PBCL, acetal-PEO-b-PCCL and acetal-PEO-b-PCL-DOX into aldehyde group

The acetal group on the surface of the polymeric micelles was converted to aldehyde groups by drop-wise addition of 0.5 mol/L HCl at room temperature adjusting the pH of the medium to 2. After stirring for 2 h, the mixture was neutralized with NaOH (0.5 mol/L) to stop the reaction. The degree of aldehyde group substitution at the end of PEO chain on the micellar shell was estimated comparing the peak intensity of the aldehyde proton (HC═O, δ=9.8) to that of PEO (CH₂CH₂, δ=3.65).

The acetal group on the micellar surface was converted into aldehyde with the yield of 70%.

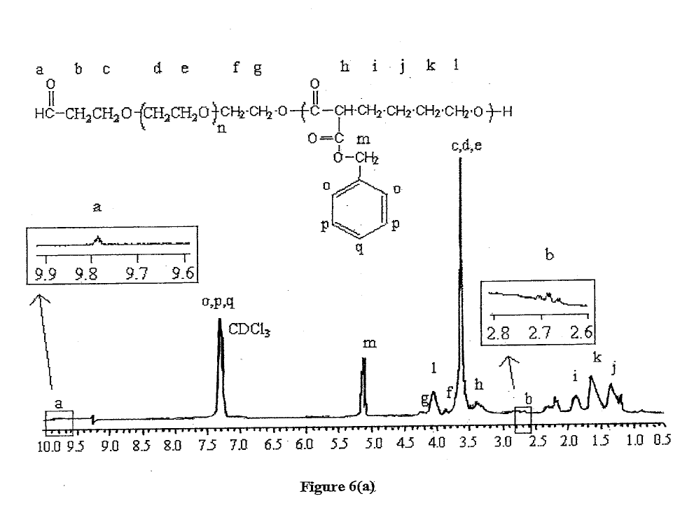

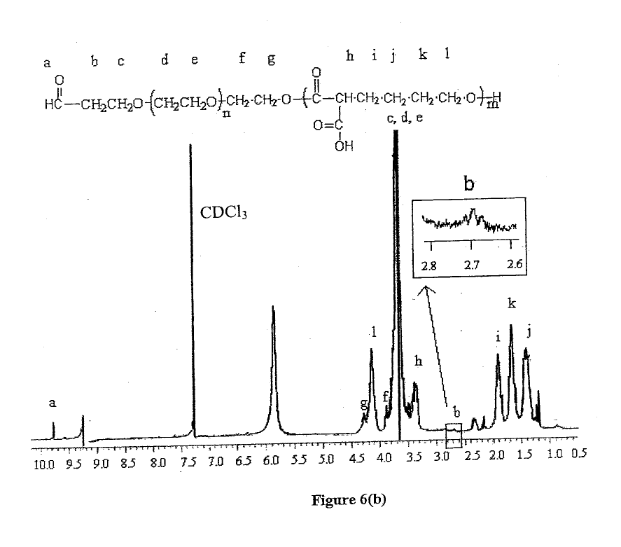

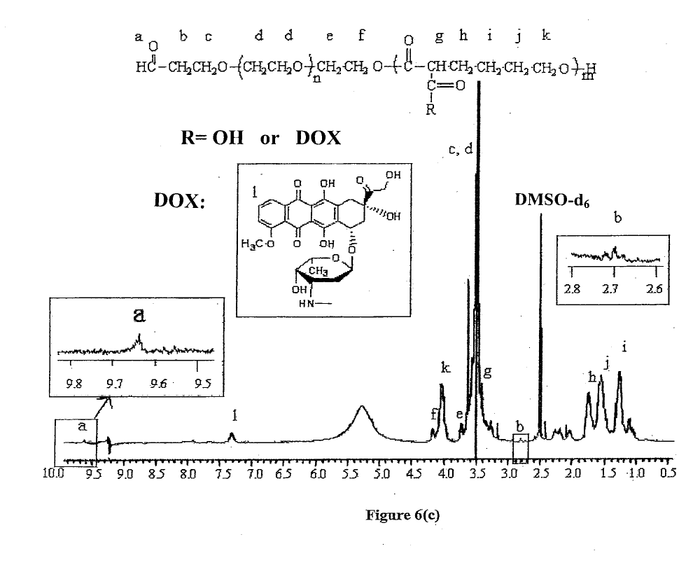

¹H NMR (CDCl₃) at 300 MHz for aldehyde-PEO-b-PBCL: δ: 1.25-2.0 (m, 6H); δ: 3.65 (s, 4H); δ: 4.10 (tri, 2H); δ: 5.15 (s, 2H); δ: 7.0-7.5 (s, 5H); δ: 9.78 (s, 1H) (FIG. 6A);

¹H NMR (CDCl₃) at 300 MHz for aldehyde-PEO-b-PCCL: δ: 1.25-2.0 (m, 6H); δ: 3.65 (s, 4H); δ: 4.10 (tri, 2H); δ: 9.78 (s, 1H) (FIG. 6B);

¹H NMR (CDCl₃) at 300 MHz for aldehyde-PEO-b-PCL(DOX): δ: 1.25-2.0 (m, 6H); δ: 3.65 (s, 4H); δ: 4.05 (tri, 2H); δ: 7.35 (s, 3H); δ: 9.65 (s, 1H) (FIG. 6C).

The molecular weight of prepared aldehyde-PEO-b-PBCL, aldehyde-PEO-b-PCCL and aldehyde-PEO-b-PCL(DOX) block copolymer measured by comparing the peak intensity of PEO (—CH₂CH₂O—, δ: 3.65) to that of methylene protons (—CH₂O, δ: 4.0-4.2) of the PBCL, PCCL and PCL(DOX) in the ¹H NMR spectra was calculated to be 6660, 5050 and 5200 g/mol⁻¹, respectively (Table 1).

### Example 10

### Validation of the Functionality of Aldehyde Group of the Polymer

Amine-containing phenylalanine (Phe) and aldehyde-PEO-b-PCL were used to test the functionality of aldehyde group and establish the protocol for peptide conjugation to the polymers. Sodium phosphate buffer (pH=7.0, ionic strength 0.1 M) solution was added to the aldehyde-terminated PEO-b-PCL micelles to obtain a 4 mg/mL polymer concentration. Phe was added and incubated with the polymeric micelles at 1:2 molar ratio (Phe:aldehyde-terminated block copolymers) at room temperature for 2 h under moderate stirring. After 2 h, NaBH₃CN (10 eq.) was added to the polymer to reduce the Schiff base. Unreacted Phe and reducing reagent were removed from the solution by gel filtration using a column packed with Sephadex G-50 (Pharmacia Biotech, Germany) followed by dialysis against water. The resulting Phe-PEO-b-PCL micelles were then lyophilized and stored at −20° C. until use. The freeze-dried sample was redissolved in DMSO-d₆ (40 mg/mL) to measure the ¹H NMR spectra. The yield of the conjugation for Phe per polymer chain was determined from the peak intensity ratio of the aromatic protons in Phe (δ=7.1-7.3) to the methene proton (—CH₂O—, δ=4.05 ppm) in the PCL segment of the block copolymer.

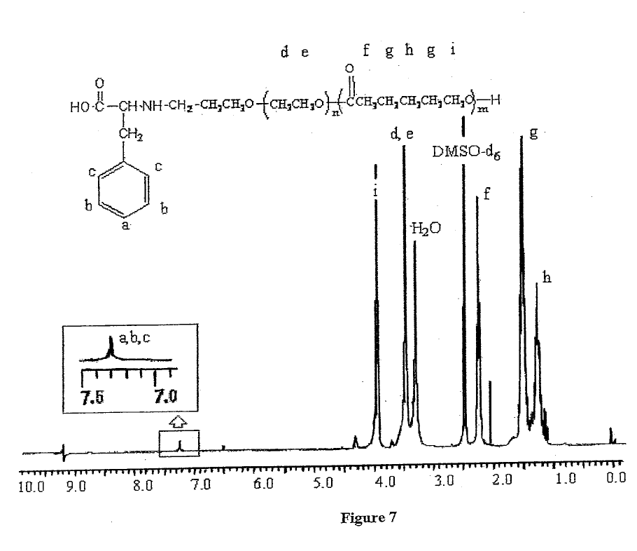

Aldehyde-PEO-b-PCL was used to validate the functionality of the aldehyde groups on the polymers in reaction with amino acid. The result is shown in FIG. 7. The aldehyde proton completely disappeared and aromatic protons of Phe appeared in the ¹H NMR spectra of the reaction product (a, b and c), indicating the chemical conjugation of Phe into the end of the PEO chain of the micelle.

### Example 11

### Peptide Conjugation

Using the established protocol, GRGDS, RGD4C and P160 were conjugated to the polymers, respectively (see Scheme 4 below).

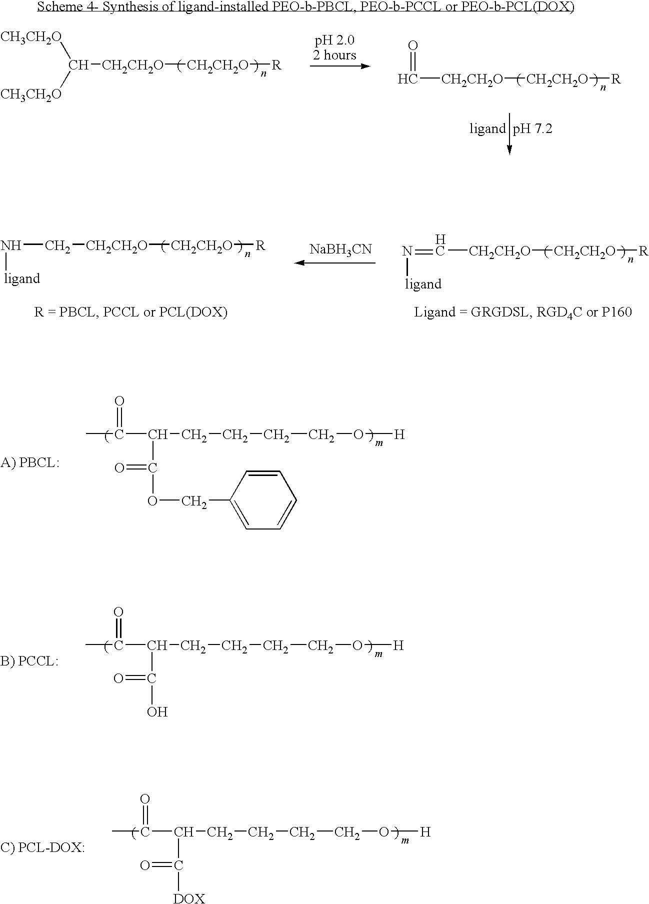

The chemical structure of GRGDS-PEO-b-PCL(DOX) (A), RGD4C-PEO-b-PCL(DOX) (B) and P160-PEO-b-PCL(DOX) (C) is shown in Scheme 5.

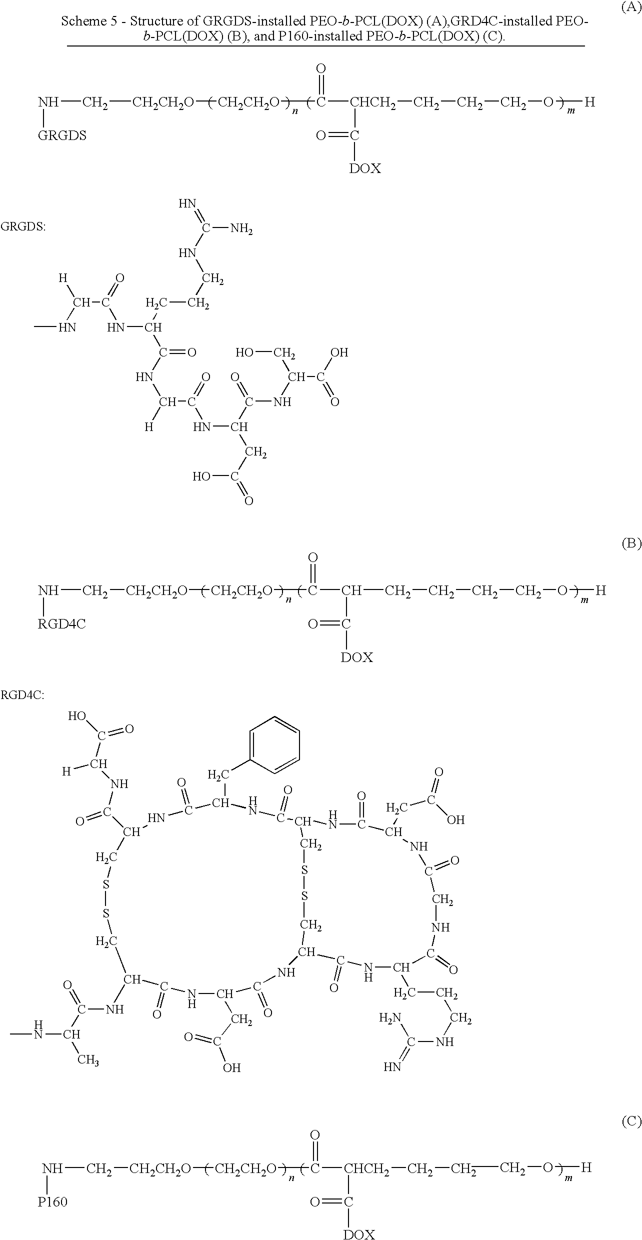

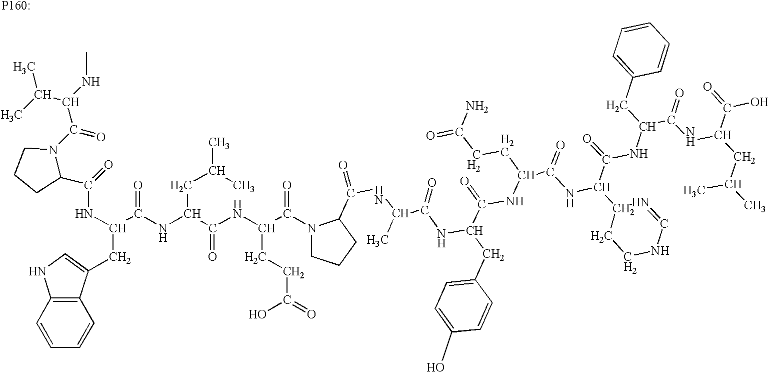

Briefly, sodium phosphate buffer (pH=7.0, ionic strength 0.1 M) solution was added to the aldehyde-terminated micelles to obtain a 4 mg/mL polymer concentration. GRGDS, p160 or GRGD4C was incubated with the polymeric micelles at room temperature for 2 h under moderate stirring. After 2 h, NaBH₃CN (10 eq.) was added to the polymer to reduce the Schiff base. Unreacted peptide and reducing reagent were removed from the solution by gel filtration using a column packed with Sephadex G-50 (Pharmacia Biotech, Germany) followed by dialysis against water. The gradient reverse HPLC method was developed to determine the peptide conjugation efficiency.

To determine the conjugation efficiency of GRGDS, a μBondapak™ (Waters Corp., USA) C-18 analytical column (10 μm, 3.9×300 mm) was used and the reaction mixture (20 μL) was directly injected into the system in duplicate at different time points. Gradient elution was performed at a flow rate of 1 mL/min (model 600 pump, Waters, Billerica, Mass.) with the mobile phases of 0.1% TFA in H₂O (solution A) and 0.1% TFA in 90/10 acetonitrile/H₂O (solution B). The mobile phase was programmed as follows: (1) 100% A for the first 1 min, (2) a linear gradient from 100% A to 60% A in 20 min, (3) a linear gradient from 60% A to 0% A for 4 min, (4) 0% A for 2 min, (5) 0% A to 100% A in 4 min, and (6) 100% A for 5 min. The peptide elution (at 4.28-5.40 min) was detected with a Waters UV detector at 214 nm. The peptide content of the conjugates was calculated on the basis of a calibration curve of known concentrations of the peptide in the phosphate buffer. After 4 days of reaction, the micellar solution was dialyzed against water in a dialysis bag (NWCO: 3000 Dalton) to remove the possible unreacted peptide and reducing reagent.

To determine the conjugation efficiency of GRGD4C, a μBondapak™ (Waters Corp., USA) C-18 analytical column (10 μm, 3.9×300 mm) was used and the reaction mixture (20 μL) was directly injected into the system in duplicate at different time points. Gradient elution was performed at a flow rate of 1 mL/min (model 600 pump, Waters, Billerica, Mass.) with the mobile phases of 0.1% TFA in H₂O (solution A) and 0.1% TFA in 90/10 acetonitrile/H₂O (solution B). The mobile phase was programmed as follows: (1) 100% A for the first 1 min, (2) a linear gradient from 100% A to 60% A in 20 min, (3) a linear gradient from 60% A to 0% A for 4 min, (4) 0% A for 2 min, (5) 0% A to 100% A in 4 min, and (6) 100% A for 5 min. The peptide elution (at 4.28-5.40 min) was detected with a Waters UV detector at 258 nm. The peptide content of the conjugates was calculated on the basis of a calibration curve of known concentrations of the peptide in the phosphate buffer. After 4 days of reaction, the micellar solution was dialyzed against water in a dialysis bag (NWCO: 3000 Dalton) to remove the possible unreacted peptide and reducing reagent.

To determine the conjugation efficiency of P160, a μBondapak™ (Waters Corp., USA) C-18 analytical column (10 μm, 3.9×300 mm) was used and the reaction mixture (20 μL) was directly injected into the system in duplicate at different time points. Gradient elution was performed at a flow rate of 1 mL/min (model 600 pump, Waters, Billerica, Mass.) with the mobile phases of 0.1% TFA in H₂O (solution A) and 0.1% TFA in 90/10 acetonitrile/H₂O (solution B). The mobile phase was programmed as follows: (1) 80% A for the first 5 min, (2) a linear gradient from 80% A to 30% A in 30 min, (3) a linear gradient from 30% A to 0% A in 5 min, (4) 0% A for 5 min, (5) 0% A to 80% A in 5 min. The peptide elution (at 4.28-5.40 min) was detected with a Waters UV detector at 258 nm. The peptide content of the conjugates was calculated on the basis of a calibration curve of known concentrations of the peptide in the phosphate buffer. After 4 days of reaction, the micellar solution was dialyzed against water in a dialysis bag (NWCO: 3000 Dalton) to remove the possible unreacted peptide and reducing reagent.

Finally, the resulting peptide-attached polymeric micelles were lyophilized and stored at −20° C. until use.

Amino acids with an aromatic residue, e.g. phenylalanine (Phe), were first chosen to react with the aldehyde group on the aldehyde-PEO-b-PCL micelles for conventionally determining the reactivity from ¹H NMR. The ¹HNMR of Phe-attached PEO-b-PCL (Phe-PEO-b-PCL) was shown in FIG. 7. Obviously, the aldehyde proton completely disappeared with the appearance of protons assignable to the aromatic residue of Phe (a, b and c), indicating the chemical conjugation of Phe to the end of the PEO chain of the micelles. Phe conjugation in the reaction was calculated to be 44 mol %/polymer from the ¹H NMR spectrum.

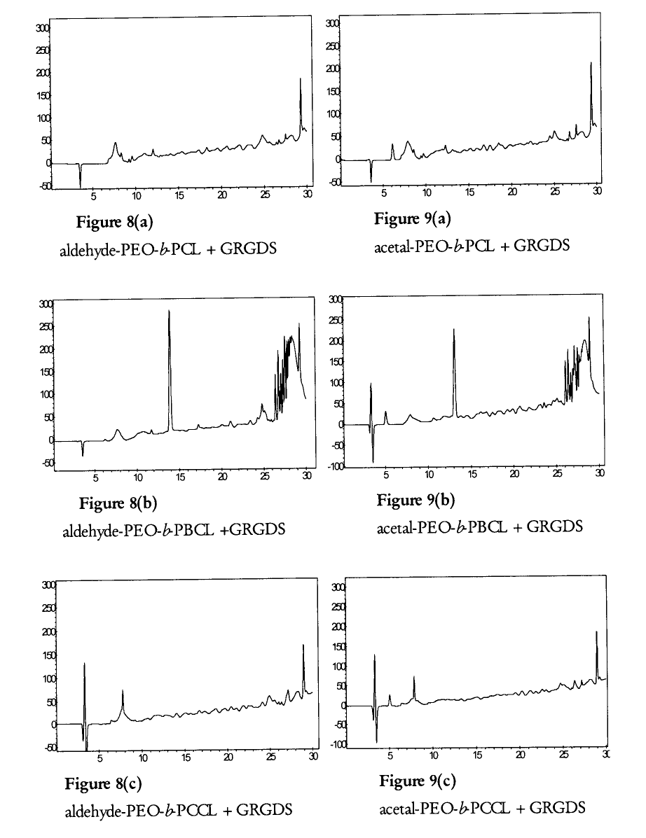

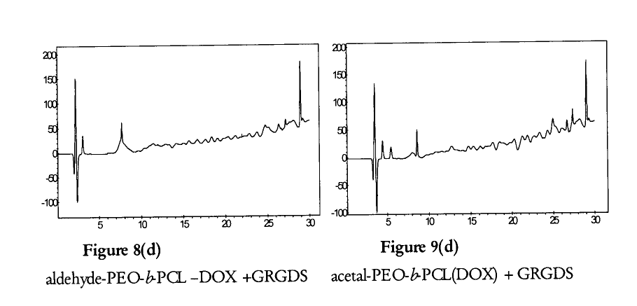

Reverse-phase HPLC method was further established to quantify the conjugation efficiency by measuring the reduction of the free peptide in the micellar solution. For the peptide conjugation, GRGDS and the aldehyde-terminated polymeric micellar solutions were incubated at a 1:2 molar ratio. Meanwhile, GRGDS and acetal-terminated polymeric micellar solutions were incubated at the same molar ratio as the control. A clear retention peak at 5.4-6.2 min could be obtained under the elution conditions described in the Experimental section without any interference. With a set of GRGDS standards, the peak area was correlated (R²=0.9998) with the injected peptide concentration. As expected, the GRGDS in the aldehyde-terminated micellar solution was completely disappeared after 60 h reaction (FIG. 8. A-D). In contrast, there is no significant change in the GRGDS concentration for the acetal-terminated micellar solutions after 72 h of reaction (FIG. 9. A-D). The peptide conjugation efficiency was calculated at 50% when the feed ratio of peptide to polymer was at 1:2 (peptide:polymer, molar ratio).

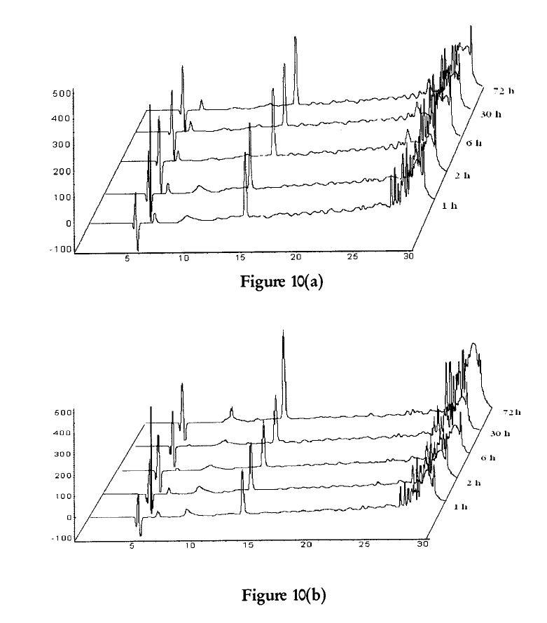

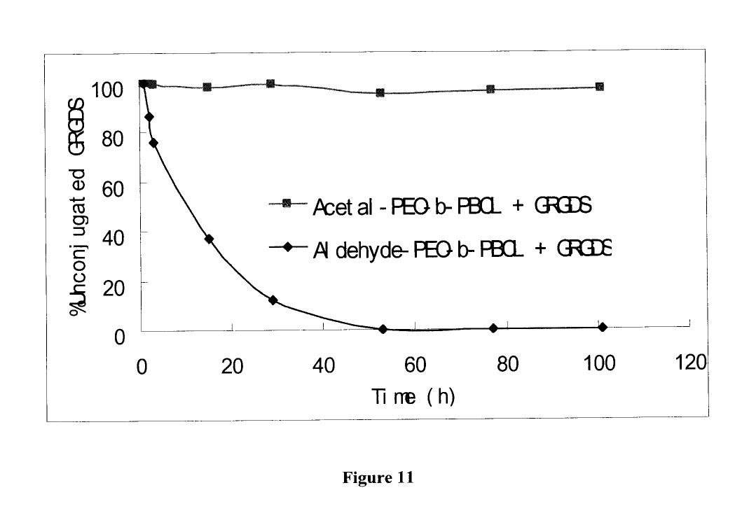

To investigate the kinetics of peptide conjugation, acetal-PEO-b-PBCL and aldehyde-PEO-b-PBCL micelles were incubated with free GRGDS at a 1:2 molar ratio (GRGDS:Polymer). At desired time points, an aliquot of the reaction mixture was sampled and injected into the C-18 column to quantify the amount of the unconjugated GRGDS in the reaction mixture. The HPLC spectrum were displayed (FIGS. 10a and 10b) and plotted (FIG. 11). As time progressed, the GRGDS peak decreased rapidly with the 30 h of incubation and completely disappeared after 72 h when GRGDS was incubated with aldehyde-PEO-b-PBCL micelles, indicating the successful conjugation of GRGDS to the micelles. However, the GRGDS peak didn't have significant change when the GRGDS was incubated with acetal-PEO-b-PBCL micelles.

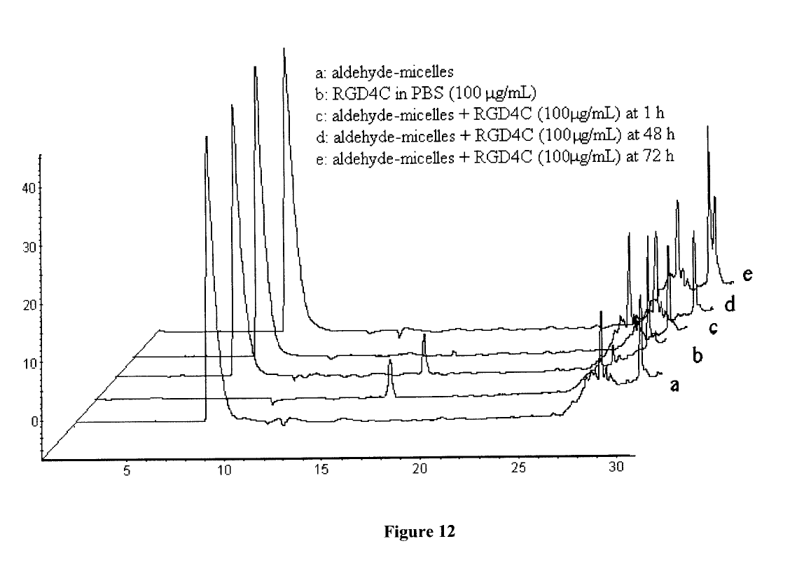

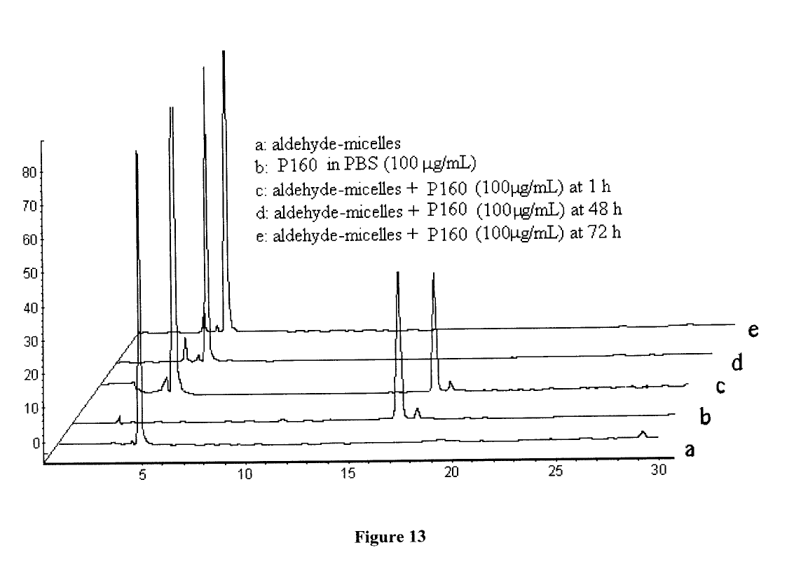

RGD4C (100 μg/mL) and P160 (100 μg/mL) were then incubated with aldehyde-PEO-b-PCL(DOX) micelles (20 mg/mL), respectively, and their conjugation efficiency was determined by HPLC. Under the reaction condition, RGD4C and P160 were consumed completely and the conjugation efficiency was calculated to be 2.5% and 1.7% polymer (molar ratio), respectively (FIG. 12 and FIG. 13).

### Example 12

### Preparation of Doxorubicin-Loaded Polymeric Micelle

Polymeric micelles containing physically loaded DOX were prepared by co-solvent evaporation method. In brief, 10 mg of acetal-PEO-b-PCL, acetal-PEO-b-PCBCL, acetal-PEO-b-PCCL, GRGDS-PEO-b-PCL GRGDS-PEO-b-PBCL or GRGDS-PEO-b-PCCL and 2 mg of free DOX were dissolved in acetone (2 mL) in a glass vial followed by addition of 3.0 equivalent of triethylamine (TEA). This solution was added dropwise to pure water (6 mL) under moderate stirring at 25° C. The micellar solution was left stirring overnight, allowing evaporation of acetone. The residual acetone was completely removed by evaporation under vacuum. DOX-loaded micellar solution was then dialyzed against a large quantity of water for 8 h using a pre-swollen semi-permeable membrane (Spectra/Pro 6, molecular weight cut-off 3000, SECTRUM, Houston, USA) to remove free DOX. The concentration of loaded DOX was quantified photometrically at 485 nm after redissolving the freeze-dried sample in a DMSO-CHCl₃ mixture (1:1, v/v).

Characteristic properties of acetal-PEO-b-PCL(DOX), acetal-PEO-b-PBCL, acetal-PEO-b-PCCL, acetal-PEO-b-PCL(DOX), GRGDS-PEO-b-PCL, GRGDS-PEO-b-PBCL, GRGDS-PEO-b-PCCL and GRGDS-PEO-b-PCL(DOX) micelles with or without loaded DOX were measured as shown in Table 2. It is noteworthy to mention that precipitation was observed when free DOX and acetal- or GRGDS-PEO-b-PCCL solution in acetone was added into the water to prepare the DOX-loaded micelles. Also aggregation and precipitation happened for DOX loaded acetal- and GRGDS-PEO-b-PCL micelles during dialysis.

### Example 13

### Cytotoxicity Experiments

Microculture tetrazolium (MTT) method was used to evaluate the cytotoxicity of DOX-incorporated polymeric micelles against B16-F10 cells. Briefly, 4000 cells were plated in 96-well plates and incubated for 24 h to allow the cells to attach. Thus, the cells were exposed to serial concentrations of free, polymeric micellar encapsulated or conjugated DOX at 37° C. for 24 or 48 h, followed by addition of 20 μL of MTT solution and incubation for another 3 h. Living cells metabolize MTT to a dark formazan dye. The cell culture media was removed by aspiration and replaced with 200 μL DMSO. The absorbance was measured spectrophotometrically using a microplate reader at dual wavelengths of 570 nm and 650 nm. The data reported represent the means of triplicate measurement.

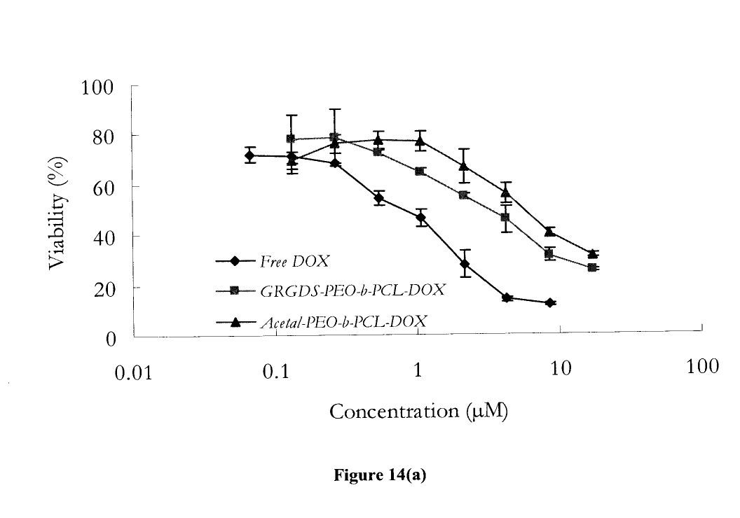

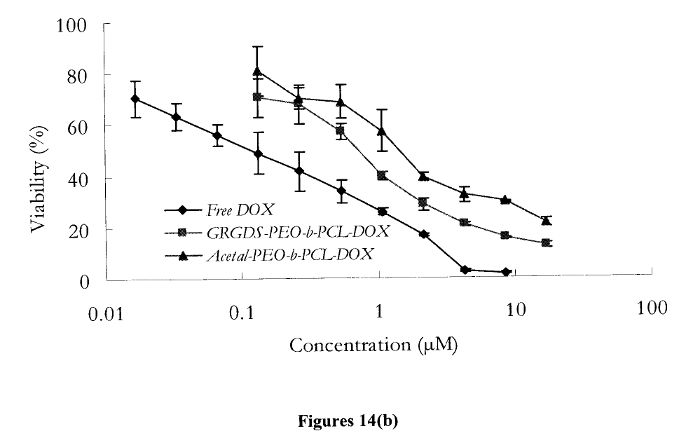

The dose-dependent growth inhibitory effect of free DOX and polymeric micelles containing encapsulated and conjugated DOX on B16-F10 cells was assessed. Cells were exposed to various DOX concentrations (either free DOX or DOX equivalent for DOX loaded micelles or polymer-drug conjugates) to determine the concentration necessary to inhibit the tumor cell growth by 50% relative to non-treated control cells (IC₅₀). Each ICₛₒ value was analyzed by polynomial curve fitting of cell viability (%) vs. DOX equivalent concentration. FIGS. 14a and 14b clearly illustrated dose-dependence of cell viability for free and conjugated DOX on a logarithm scale and indicated a significant increase in the cytotoxicity of conjugated DOX with GRGDS modified micelles compared to acetal-micelles. The IC₅₀ of DOX for all formulations is summarized in Table 3. From these results, it is evident that covalent attachment of DOX to the polymer carrier decreases the cytotoxicity of the parent drug, significantly. The IC₅₀ of acetal-PEO-b-PCL(DOX), was 14.3- and 6.3-fold higher than free DOX after 24 and 48 h incubation, respectively. The difference between the IC₅₀ of free and polymeric micellar DOX conjugates reflects different mechanisms of cell uptake, i.e., diffusion for free DOX vs. endocytosis for polymeric micellar DOX conjugates, resulting in different extent and rate of intracellular drug concentrations. Conjugation of GRGDS to the micellar surface significantly increased the cytotoxicity of polymeric micellar DOX conjugates, although it still possessed lower cytotoxicity than free DOX. After 24 and 48 h incubation, GRGDS-PEO-b-PCL(DOX) micelles only showed a 7.9 and 3.3-fold decrease in the IC₅₀ in comparison to free DOX, respectively. This might be due to facilitated accumulation of polymeric micellar DOX conjugate by receptor mediated endocytosis leading to improved drug cleavage from polymer in the endosomes and lysosomes.

For the physically DOX-loaded micelles, there was no significant difference observed between the cytotoxicity of DOX loaded in acetal-PEO-b-PCL and GRGDS-PEO-b-PCL or acetal-PEO-b-PCCL and GRGDS-PEO-b-PCCL. Only GRGDS-PEO-b-PBCL showed a 1.9-fold decrease in IC₅₀ compared to acetal-PEO-b-PBCL. The highest IC₅₀ (lowest efficacy) was observed with GRGDS-PEO-PEO-b-PCCL and acetal-PEO-b-PCCL. The larger size of particles in this case might have a negative effect on the ligand-receptor recognition or on the cellular uptake of GRGDS-PEO-PEO-b-PCCL and acetal-PEO-b-PCCL micelles.

TABLE 1
Characteristics of polymers

| | | | | | | PEO end |
|---|---|---|---|---|---|---|
| | | Theoretical | | Polymer | | group/polymer |
| | PEO Mₙ^{a} | PCL Mw | PCL Mₙ^{a} | Mₙ^{b} | Polydispersity | conjugation |
| Polymer | (g · mol.⁻¹) | (g · mol.⁻¹) | (g · mol.⁻¹) | (g · mol.⁻¹) | (M_{w}/Mₙ)^{b} | level |
| Acetal-PEO | 3600 | — | — | 3320 | 1.340 | 100%^{a} |
| Acetal-PEO-b-PCL | 3600 | 5000 | 1460 | 5160 | 1.458 | 100%^{a} |
| Aldehyde-PEO-b-PCL | 3600 | 5000 | 1380 | 4830 | 1.350 | 33.8%^{a} |
| GRGDS-PEO-b-PCL | 3600 | 5000 | 1390 | 4910 | 1.467 | 50.0%^{c} |
| Acetal-PEO-b-PBCL | 3600 | 3600 | 3060 | 5240 | 1.639 | 100%^{a} |
| Aldehyde-PEO-b-PBCL | 3600 | 3600 | 3410 | 5100 | 1.738 | 30.1%^{a} |
| GRGDS-PEO-b-PBCL | 3600 | 3600 | 3520 | 4980 | 1.459 | 50.0%^{d} |
| Acetal-PEO-b-PCCL | 3600 | 2290 | 1470 | 4070 | 1.410 | 95.6%^{a} |
| Aldehyde-PEO-b-PCCL | 3600 | 2290 | 1450 | 3870 | 1.296 | 74.6%^{a} |
| GRGDS-PEO-b-PCCL | 3600 | 2290 | 1480 | 3970 | 1.310 | 50.0%^{d} |
| Acetal-PEO-b- | 3600 | 6270 | 1850 | 5130 | 1.414 | 100%^{a} |
| PCL(DOX) | | | | | | |
| Aldehyde-PEO-b- | 3600 | 6270 | 1700 | 5200 | 1.368 | 29.8%^{a} |
| PCL(DOX) | | | | | | |
| GRGDS-PEO-b- | 3600 | 6270 | 1760 | 5250 | 1.351 | 50.0%^{d} |
| PCL(DOX) | | | | | | |

^{a}Determined by ¹H NMR;
^{b}Determined by GPC;
^{c}Determined by GPC-UV detection at 214 nm;
^{d}Estimated based on conjugation efficiency of GRGDS-PEO-b-PCL

TABLE 2
Characteristics of the copolymer micelles with or without physically loaded DOX.

| | Empty micelles | | | DOX-loaded micelles | | | |
|---|---|---|---|---|---|---|---|
| | Average | | | Average | | Drug/polymer | |
| | diameter | Poly- | | diameter | Poly- | Loading | |
| Polymer | (nm) | dispersity | Stability^{a} | (nm) | dispersity | (w/w) | Stability^{a} |
| Acetal-PEO-b-PCL | 93.0 | 0.22 | >1 month | 148.6 | 0.15 | 2.81 | <24 hours |
| GRGDS-PEO-b-PCL | 99.6 | 0.19 | >1 month | 144.2 | 0.48 | 2.79 | <24 hours |
| Acetal-PEO-b-PBCL | 69.1 | 0.27 | >1 month | 68.4 | 0.28 | 3.78 | >3 weeks |
| GRGDS-PEO-b- | 72.3 | 0.31 | >1 month | 69.6 | 0.19 | 3.42 | >3 weeks |
| PBCL | | | | | | | |
| Acetal-PEO-b-PCCL | 48.0 | 0.37 | >1 month | 599.4 | 0.36 | 3.04 | <24 hours |
| GRGDS-PEO-b- | 49.2 | 0.25 | >1 month | 600.5 | 0.72 | 2.98 | <24 hours |
| PCCL | | | | | | | |
| Acetal-PEO-b- | 90.4 | 0.34 | >1 month | — | — | — | — |
| PCL(DOX) | | | | | | | |
| GRGDS-PEO-b- | 89.4 | 0.45 | >1 month | — | — | — | — |
| PCL(DOX) | | | | | | | |

^{a}Stability was measured based on the appreciable aggregation or precipitation of the micellar solution in water.

TABLE 3
Cytotoxicity of DOX-containing micelles
against B16-F10 cells in vitro.

IC₅₀ (μM) of
DOX equivalent

| Formulations | 24 h | 48 h |
|---|---|---|
| Free DOX | 0.417 | 0.204 |
| Acetal-PEO-b-PCL/DOX | 1.183 | 0.563 |
| GRGDS-PEO-b-PCL/DOX | 1.068 | 0.526 |
| Acetal-PEO-b-PBCL/DOX | 3.117 | N.D. |
| GRGDS-PEO-b-PBCL/DOX | 1.636 | N.D. |
| Acetal-PEO-b-PCCL/DOX | 9.706 | N.D. |
| GRGDS-PEO-b-PCCL/DOX | 10.943 | N.D. |
| Acetal-PEO-b-PCL(DOX) | 5.947 | 1.285 |
| GRGDS-PEO-b-PCL(DOX) | 3.271 | 0.662 |

N.D. not determined

### Example 14

### Synthesis of Fmoc-hydrazine conjugated acetal-poly(ethylene oxide)-block-poly(caprolactone) (acetal-PEO-b-P(CL-Hydrazone-Fmoc)) block copolymer

Fmoc-protected hydrazine was synthesized as described in Zhang, R. E., Cao, Y. L., Hearn, M. W., Synthesis and application of Fmoc-hydrazine for the quantitative determination of saccharides by reversed-phase high-performance liquid chromatography in the low and subpicamole range, Anal Biochem. (1991) 195(1): 160-7, incorporated herein by reference. Then Fmoc-hydrazine was conjugated to the acetal-PEO-b-PCCL by amide bond formation as shown in Scheme 6 below.

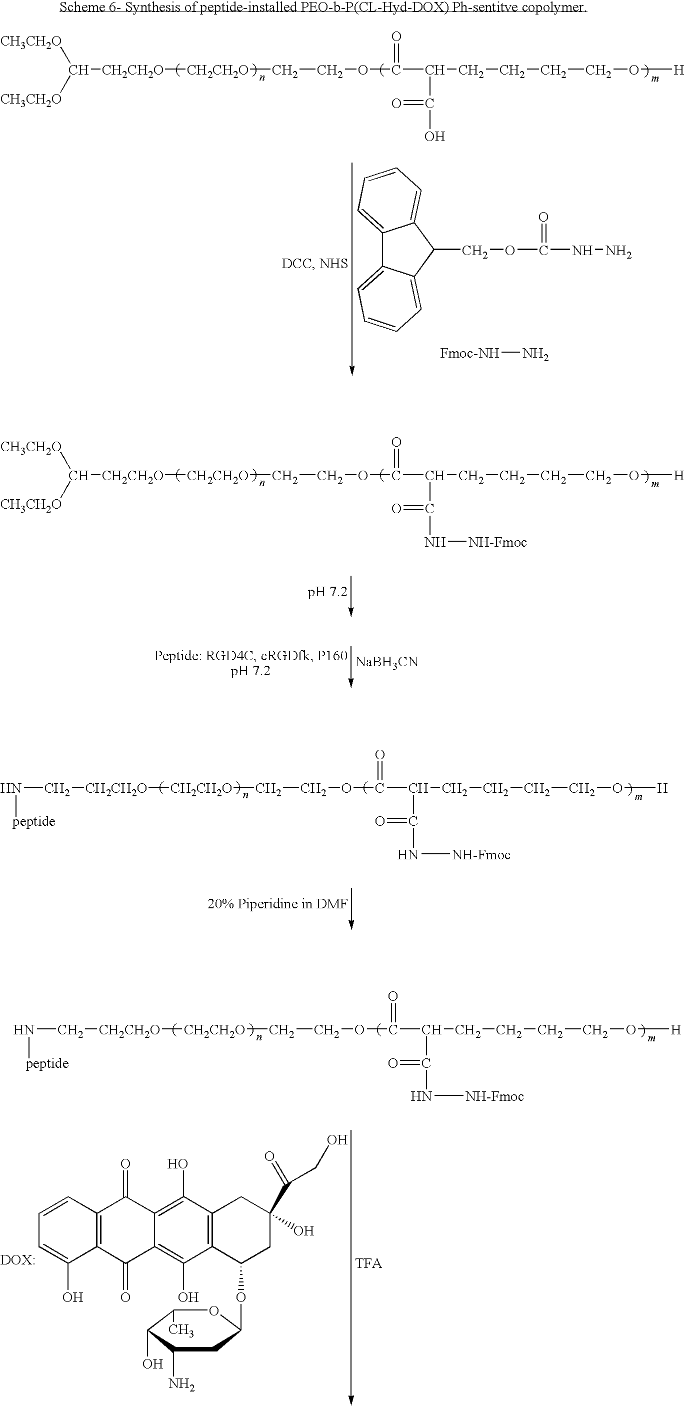

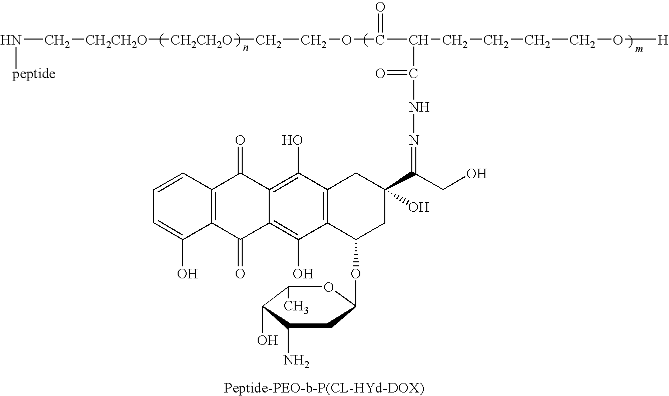

Briefly, to a solution of acetal-PEO-b-PCCL (150 mg, ˜0.015 mmol) in 30 mL of dry THF, 1,3-dicyclohexycarbodiimide (DCC) (28.75 mg, 0.25 mmol) and N-hydroxysuccinimide (NHS) (51.5 mg, 0.25 mmol) in tetrahydrofuran was added. The reaction was allowed for 5 h till precipitate was formed. Then, Fmoc-hydrazine (63.5 mg, 0.25 mmol) dissolved in THF was added drop-wise to the polymer solution. The reaction proceeded for another 24 h in room temperature. The resulting solution was centrifuged to remove the precipitate followed by evaporation under vacuum to remove the solvents. 10 mL of methanol was then introduced to dissolve the product. The result solution was precipitated from diethyl ether and dialyzed in DMSO with molecular weight cut (MWCO) 1000 membranes fro further purification. After substituting DMSO with distilled water, the product was freeze-dried and stored under 4° C. for further use.

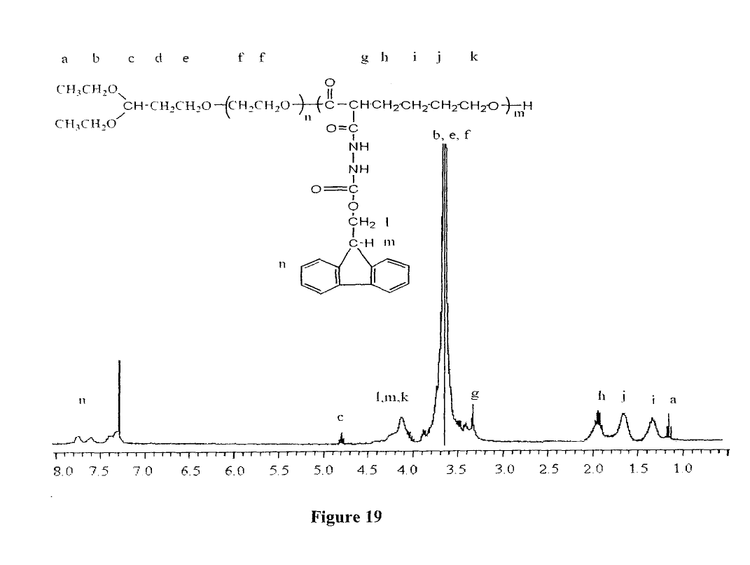

The yield for the preparation of block copolymer was 86%. ¹H NMR (DMSO-d₆) at 300 MHz: δ: 1.20 (tri, 6H); δ: 1.25-2.0 (m, 6H); δ: 3.65 (s, 4H); δ: 4.05 (tri, 2H); δ: 4.55 (t, 1H); δ: 7.0-7.5 (m, 8H) (shown in FIG. 19). The molecular weight (Mn) of acetal-PEO-b-P(CL-Hyd-Fmoc) based on ¹H NMR was 5740.

### Example 15

### Preparation of peptide-PEO-b-P(CL-Hyd-Fmoc) micelles

Acetal-PEO-b-P(CL-Hyd-Fmoc) micelles were prepared by solvent evaporation method. The acetal group on the surface of the polymeric micelles was converted to aldehyde groups by drop-wise addition of 0.5 mol/L HCl at room temperature adjusting the pH of the medium to 2. After stirring for 2 h, the mixture was neutralized with NaOH (0.5 mol/L) to stop the reaction. Using the established protocol, GRGDS, RGD4C, cRGDfK or P160 alone or cRGDfK and P160 mixture were added to the polymers.

Under the reaction condition, RGD4C and cRGDfK were consumed completely and the conjugation efficiency was calculated to be 2.5% and 4.8% polymer (molar ratio), respectively.

The structures of GRGDS, RGD4C, cRGDfK and P160 are shown in Scheme 7 below.

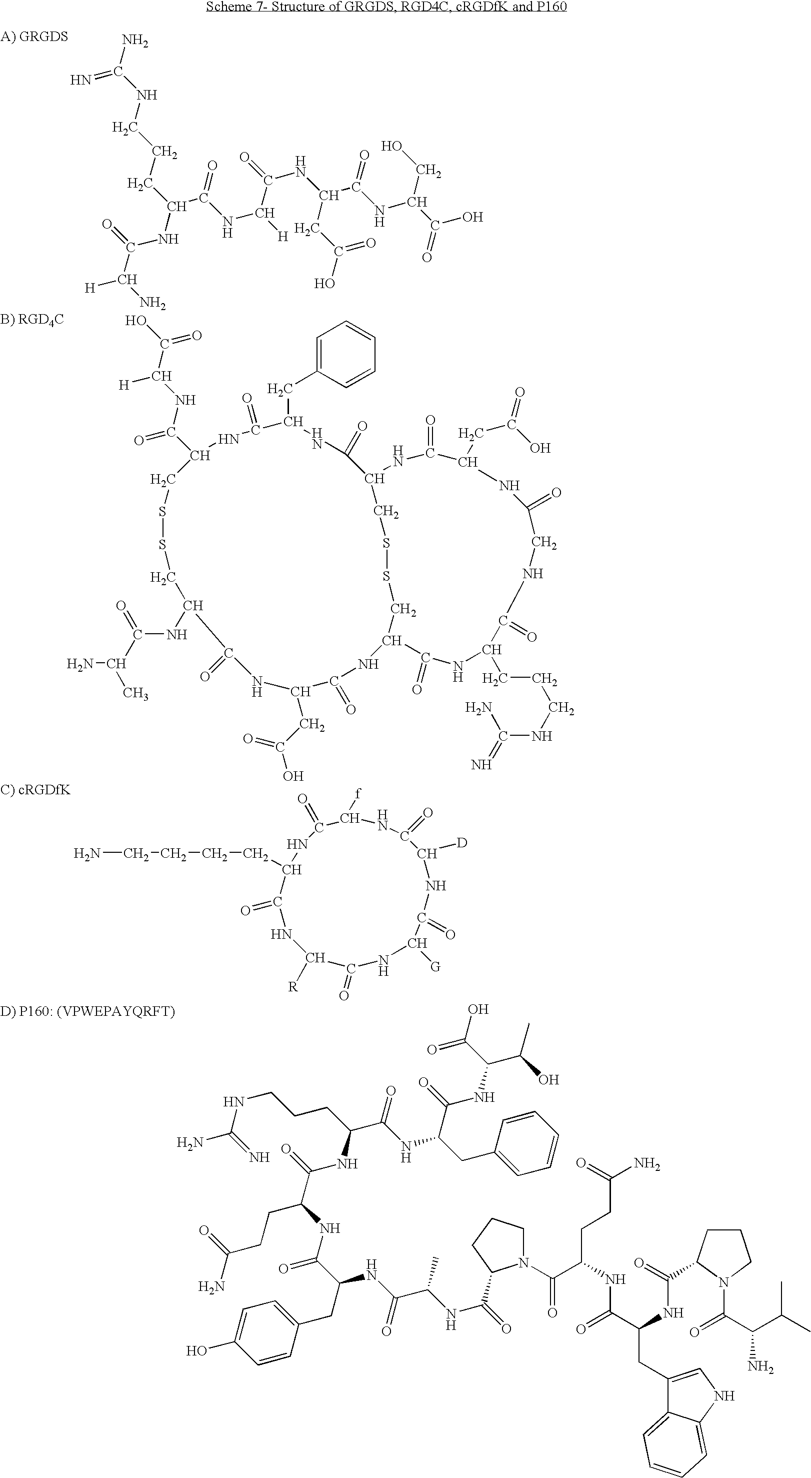

The resulting peptide-attached polymeric micelles were lyophilized and stored at −20° C. until use. ¹HNMR and GPC were used to evaluate the prepared polymers. The conjugation efficiency of peptides on polymeric micelles was evaluated by HPLC using a μBondapak™ (Waters Corp., USA) C-18 analytical column (10 μm, 3.9×300 mm) was used and the reaction mixture (20 μL) was directly injected into the system in duplicate at different time points. Gradient elution was performed at a flow rate of 1 mL/min (model 600 pump, Waters, Billerica, Mass.) with the mobile phases of 0.1% TFA in H₂O (solution A) and 0.1% TFA in 90/10 acetonitrile/H₂O (solution B). The mobile phase was programmed as follows: (1) 100% A for the first 1 min, (2) a linear gradient from 100% A to 60% A in 20 min, (3) a linear gradient from 60% A to 0% A for 4 min, (4) 0% A for 2 min, (5) 0% A to 100% A in 4 min, and (6) 100% A for 5 min. The peptide elution (at 4.28-5.40 min) was detected with a Waters UV detector at 214 nm. The peptide content of the conjugates was calculated on the basis of a calibration curve of known concentrations of the peptide in the phosphate buffer. After 4 days of reaction, the micellar solution was dialyzed against water in a dialysis bag (NWCO: 3000 Dalton) to remove the possible unreacted peptide and reducing reagent.

### Example 16

### Synthesis of doxorubicin-conjugated peptide-modified poly(ethylene oxide)-block-poly(caprolactone hydrazone DOX) (peptide-PEO-b-P(CL-Hyd-DOX)) block copolymer

Synthesized peptide-PEO-b-P(CL-Hyd-Fmoc) (250 mg) was treated with 20% piperidine DMF for 20 minutes to remove the protective Fmoc groups, precipitated in diethyl ether and dried in vacuum to obtain the polymer, peptide-PEO-b-P(CL-hydrazine). The obtained polymer (100 mg) was then dissolved in 40 mL of methanol and 20 mg of DOX-HCL dissolved in 10 mL of methanol was added with TFA as an acid catalyst. The solution was stirred at room temperature for 48 h while being protected from light till a dark orange solution formed. The resulting solution was purified by Sephadex LH column using methanol as the eluent for separating the peptide-PEO-b-P(CL-Hyd-DOX) block copolymer from the unbound free DOX. The applied solution was separated into two fractions, and the eluted first was collected. After evaporation of the methanol, the red wine color product was evaluated by RPLC to confirm the absence of unbound free DOX. The content of conjugated DOX was determined by measuring its absorbance at 485 nm, on the assumption that molar absorptivity of DOX residue bound to the polymer was identical to that of free DOX at 485 nm. DOX content was expressed in mol % with respect to the α-carboxylic-ε-caprolactone residue of acetal-PEO-b-PCCL.

The yield for the preparation of the block copolymer was 90%. The conjugation efficiency of DOX to the polymer was 33.3% (molar ratio).

### Example 17

### Drug Release

RPLC was performed to assess the pH sensitivity of the micelles. Toward this, pH-sensitive micelles with 10 mg/mL concentration were incubated under acetate buffer (pH 5.0) or phosphate buffer (pH 7.2), and their time and pH-dependent drug release profile was monitored by RPLC. Fluorescence-quenching experiment was also used to confirm the pH-sensitivity.

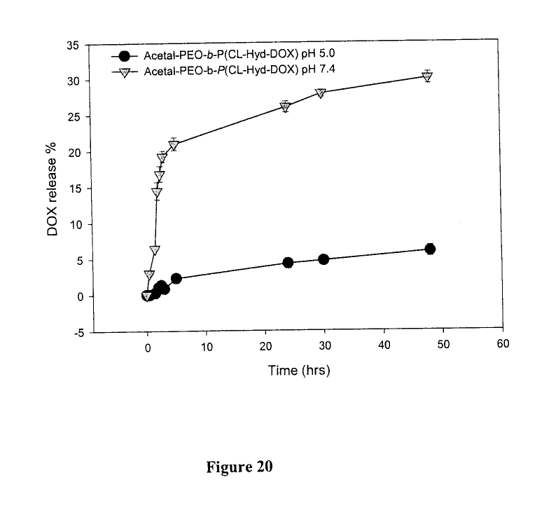

FIG. 20 illustrates that at pH 7.2 the DOX release is quite low and less than 5% of DOX was released from the micelles within 48 h of incubation. Under more acidic conditions, pH 5.0, DOX release was greatly accelerated and around 30% of the DOX was released from the micelles with 48 h of incubation.

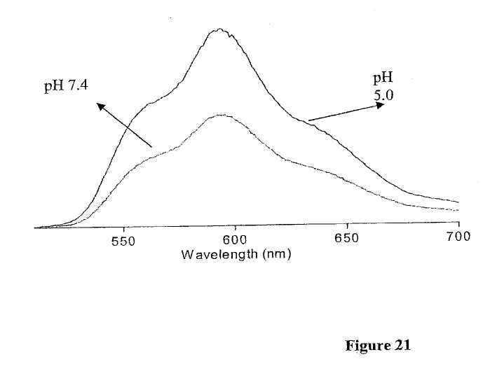

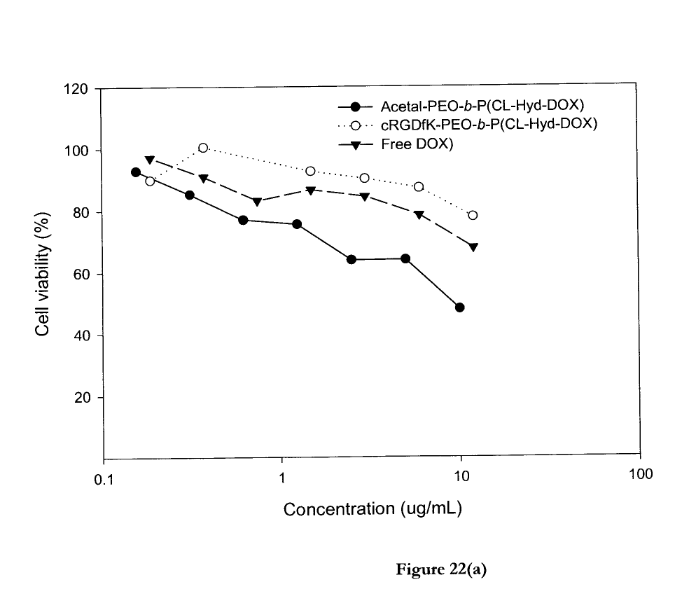

The fluorescence of DOX entrapped in the micellar core IS greatly quenched compared to free DOX. At the end of drug release, the micellar solution at pH 5.0 showed stronger DOX fluorescence than at pH 7.4, indicating DOX was effectively cleaved by acid and released from the micellar into the media. This is shown more particularly in FIG. 21.

### Example 18

### Cytotoxicity Experiments

Microculture tetrazolium (MTT) method was used to evaluate the cytotoxicity of DOX-incorporated polymeric micelles against MDA435/LCC6 sensitive or resistant cells. Briefly, 4000 cells were plated in 96-well plates and incubated for 24 h to allow the cells to attach. Thus, the cells were exposed to serial concentrations of free, pH-sensitive polymeric micellar conjugated DOX at 37° C. for 48 h, followed by addition of 20 μL of MTT solution and incubation for another 3 h. Living cells metabolize MTT to a dark formazan dye. The cell culture media was removed by aspiration and replaced with 200 μL DMSO. The absorbance was measured spectrophotometrically using a microplate reader at dual wavelengths of 570 nm and 650 nm. The data reported herein represent the means of triplicate measurement.

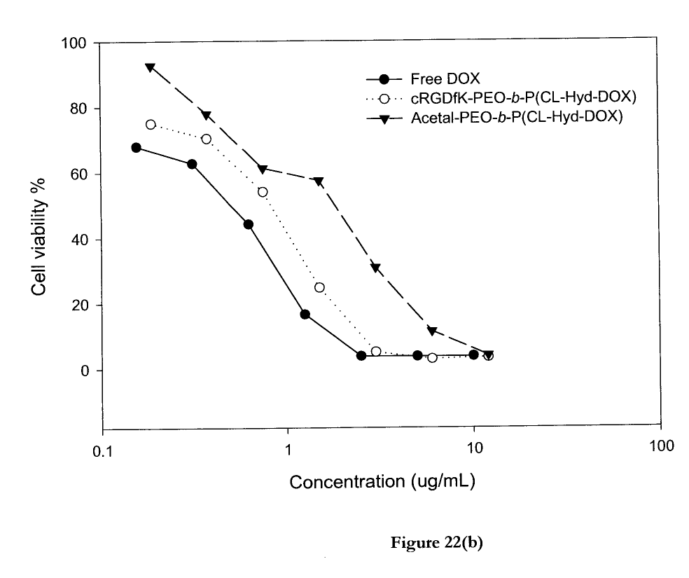

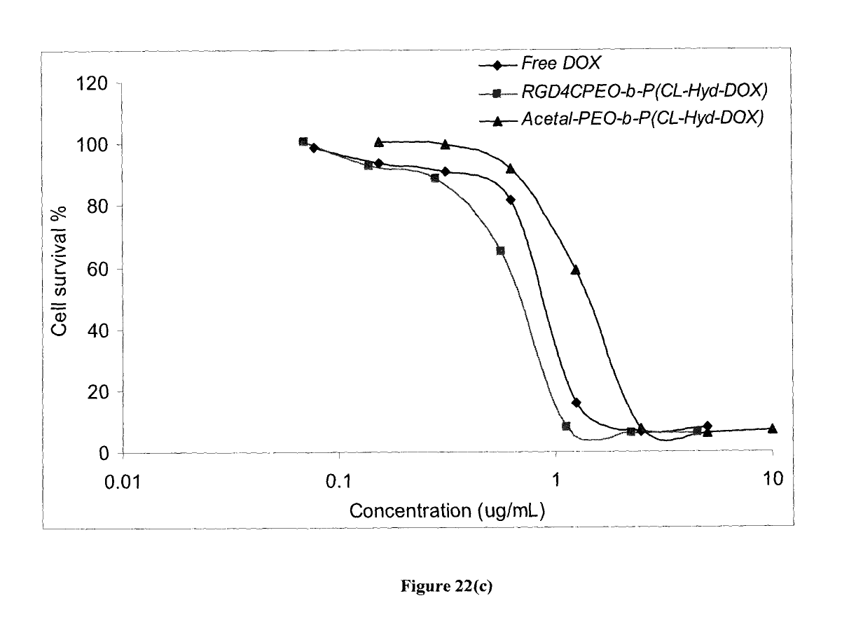

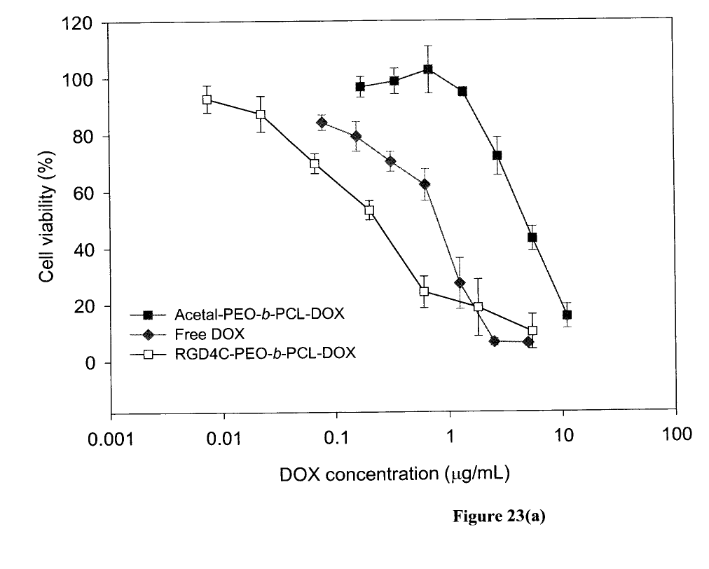

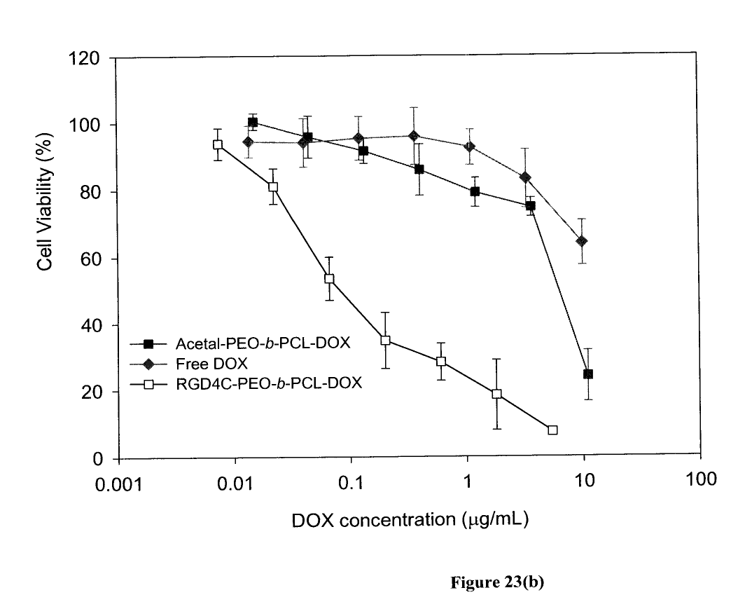

The dose-dependent growth inhibitory effect of free DOX, cRGDfK-PEO-b-P(CL-Hyd-DOX), RGD4C-PEO-b-P(CL-Hyd-DOX), and RGD4C-PEO-b-P(CL-DOX) polymeric micelles containing conjugated DOX on MDA435/LCC6 sensitive and resistant cells was assessed and the results are shown in FIGS. 22 A, B and C and FIGS. 23 A and B. Dose-dependence of cell viability for free and conjugated DOX on a logarithm scale was observed and revealed a significant increase in the cytotoxicity of conjugated DOX with cRGDfK and RGD4C-modified micelles compared to acetal-micelles.

The results of these experiments showed: (1) cRGDfK-PEO-b-P(CL-Hyd-DOX) micelles are more toxic than acetal-PEO-b-P(CL-Hyd-DOX) micelles on MDA435/LCC6 sensitive or resistant cells (FIGS. 22 A and B); (2) RGD4C-PEO-b-P(CL-Hyd-DOX) micelles are more cytotoxic than acetal-PEO-b-P(CL-Hyd-DOX) micelles on MDA435/LCC6 sensitive cells (FIG. 22C); and (3) RGD4C-PEO-b-P(CL-DOX) micelles with imide as the linker are more cytotoxic than acetal-PEO-b-P(CL-DOX) and even more toxic than free DOX against sensitive and resistant cells (FIGS. 23 A and B). The RGD4C-conjugated micelles containing chemically conjugated DOX by amide bound was more effective than even free DOX in vitro. This is a surprising results, which might be due in part to RGD4C.

### Example 19

### Evaluation of RGD4C-PEO-b-P(CL-Hyd-DOX) on SCID mice bearing MDA435/LCC6 tumors

SCID mice were inoculated with MDA435/LCC6 tumors and were then randomized with at least 7 mice per group and numbered. The tumor sizes were monitored daily during the experiment period. The tumor volume was estimated by measuring tumor width and length. Mice with size-matched tumors (0.1 cm³) were randomized into four treatment groups (n>=7): a) Control, b) free DOX, c) acetal-PEO-b-P(CL-Hyd-DOX) and d) RGD4C-PEO-b-P(CL-Hyd-DOX). Mice were treated with 2.5 mg/kg of DOX equivalent (2.5 mg/kg of polymer equivalent) by intravenous injection via tail vein on every 7th day for four doses (days 1, 7, 14, and 21). For all treatment groups, tumor size was measured on every other day with a caliper in two dimensions and calculated using the following and plotted versus time for each group:

$$Tumor weight [ 3 ] = \frac{Length ( cm ) \times {[ width ( cm ) ]}^{2}}{2} .$$

In each experiment, the mice were monitored for up to 50 days after inoculation or until one of the following conditions for euthanasia was met: (1) the mouse's body weight dropped below 15% of its initial weight; (2) the mouse's tumor was 2.0 cm across any dimension; (3) the mouse became lethargic or sick and unable to feed; or (4) the mouse was found dead. Over 50 day, all surviving mice were euthanized. Survival data is presented in a Kaplan-Meier plot.

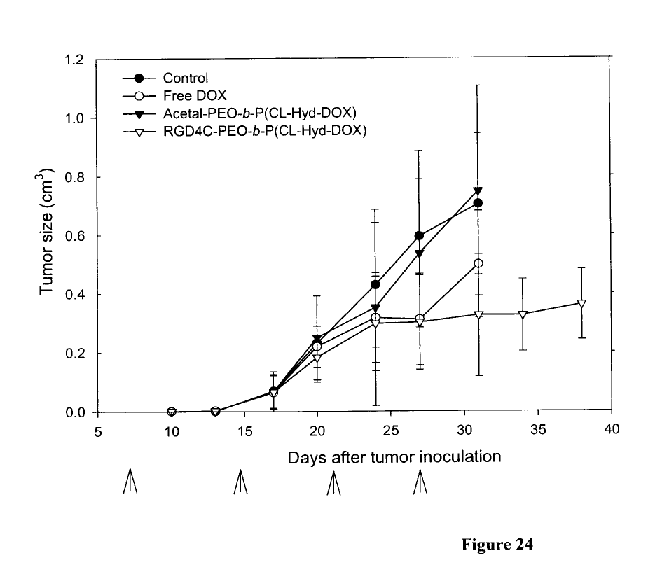

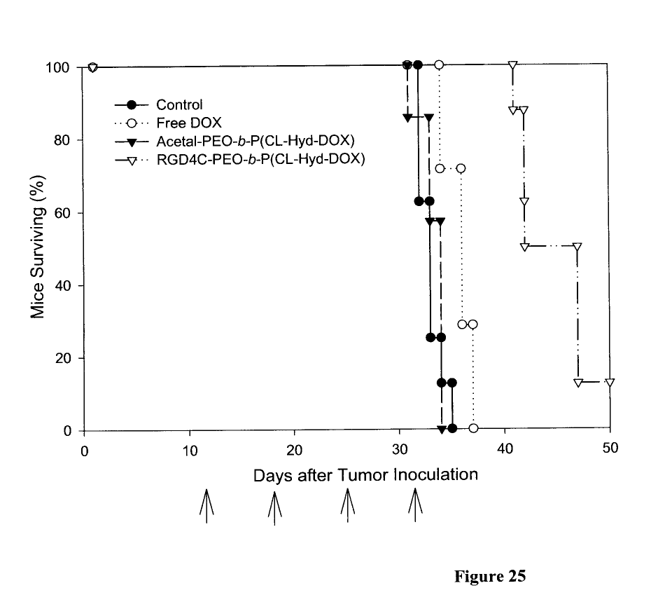

RGD4C-PEO-b-P(CL-Hyd-DOX) displayed stronger tumor growth inhibition than non-RGD4C-modified micelles or saline on SCID mice bearing MDA435/LCC6 sensitive tumors (FIG. 24). The mice treated with RGD4C-PEO-b-P(CL-Hyd-DOX) micelles showed significantly increased mean survival time (MST) compared to those received saline, free DOX or non-RGD4C modified micelles (FIG. 25).

### Example 20

### Cellular uptake of P160-modified PEO-b-PBCL micelles loaded with DiI by MDA435/LCC6 cells

### a) Florescence Spectroscopy

MDA435/LCC6 Cells were grown in RPMI 1640 complete growth medium supplemented with 10% fetal bovine serum, 1 w/v % L-glutamine, and 100 units/mL penicillin and 100 ug/mL streptomycin and were maintained at 37° C. with 5% CO2 in a tissue culture incubator. Cells were seeded into a 24-well plate (1*10⁵ cells/well) containing 1 mL of media to grow to 70% confluence after 24 h incubation. P160 or acetal-micelles containing DiI were then added and incubated with MDA-435 cells for 3 hrs at 37 or 4° C. The final DiI and polymer concentration in each well was 0.5 ug/mL and 0.5 mg/mL, respectively. Incubation at 4 and 37° C. were used to differentiate between cell binding and internalization. Free DiI was dissolved in PBS with the aid of DMSO (<1%) and was incubated with cells for 3 hrs as positive control. Samples having free and encapsulated DiI without cells and cells incubated with the medium were used as negative controls. For the competition experiments, MDA-435 cells were preincubated with excess free P160 (20 ug/mL) for 30 min to saturate receptors and to inhibit the binding and internalization of P160-conjugated micelles. Following the incubation period, medium was removed and cells were washed with cold PBS three times. Then, 1 mL of DMSO was added in each well to lyse cells. Fluorescence emission intensity of DiI at 565 nm (fluorescence concentration analyzer, Baxter, United States) provided means for the measurement of internalized DiI levels. DiI cellular accumulation was normalized with respect to total cellular protein content, which was quantified by Bradford method using bovine serum albumin (BSA) as standard. Percent uptake was calculated using the following equation:

uptake (%)=(normalized concentration of internalized DiI)*100/(concentration of encapsulated DiI added to each well).

### b) Flow Cytometry

MDA435/LCC6 cells were grown as a monolayer and harvested by 0.25% (w/v) trypsin-0.03% (w/v) EDETA solution were seeded into 6 well plate at a density (1*10⁵ cell/ml) and incubated for 24 hrs. The test samples DiI loaded P160 micelles, DiI loaded acetal micelles and free DiI were added to the well plate and were incubated for 3 hrs at 37° C. The incubation solution was removed and the cells were detached using trypsin EDTA solution and washed twice with cold PBS. The florescence of DiI internalized into the cells was measured using flow cytometer, the excitation wavelength was set at 550 nm and measured at FL2 channel.

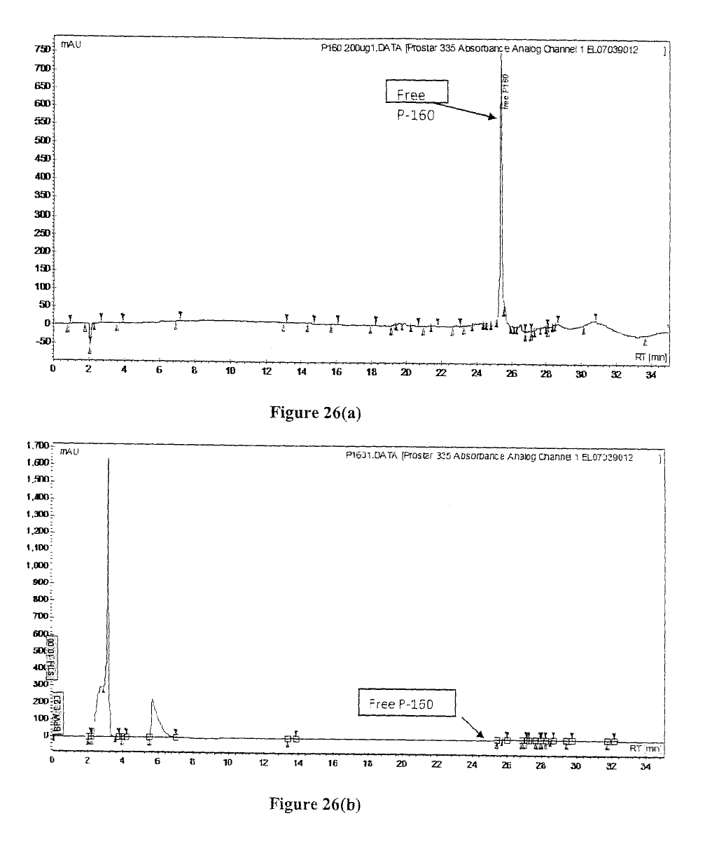

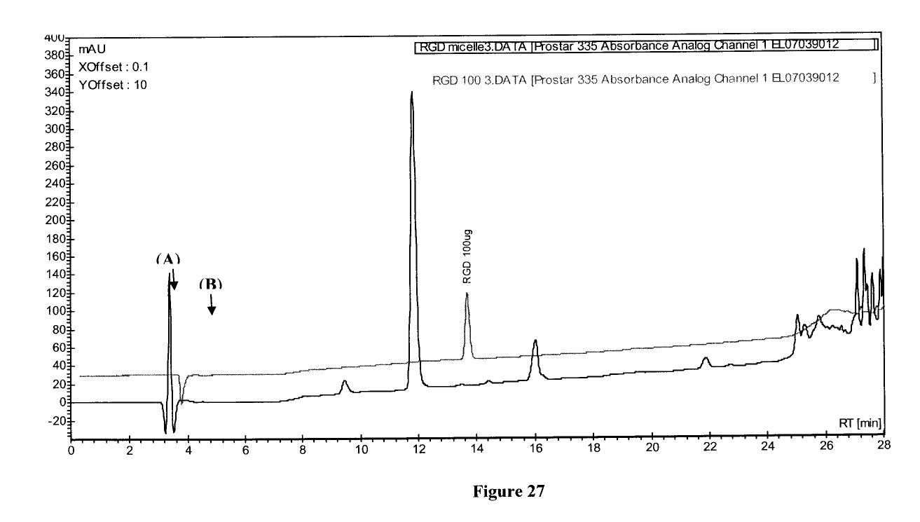

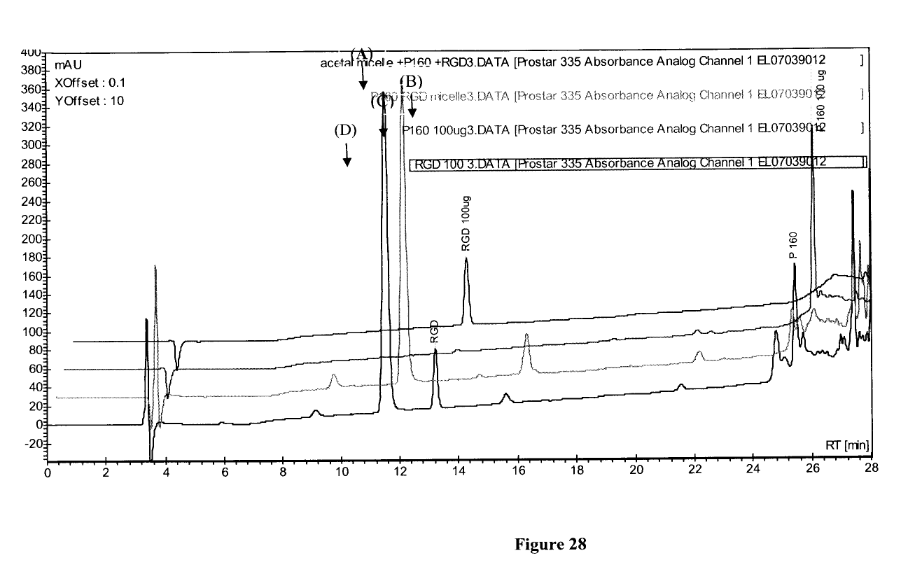

P160- and P160/GRGDfK/P160-modified micelles were prepared according to synthetic methods shown in Scheme 6. The characteristics of prepared block copolymers in summarized in Table 4. HPLC data (FIGS. 26-28) confirmed successful conjugation of the peptides to micelles as conjugation ratios detailed in Table 4 below.

TABLE 4
Characteristics of the P160-modified block copolymers

| | | | | | | PEO end |
|---|---|---|---|---|---|---|
| | | Theoretical | | Polymer | | group/polymer |
| | PEO Mₙ^{a} | PCL Mw | PCL Mₙ^{a} | Mₙ^{b} | Polydispersity | conjugation |
| Polymer | (g · mol.⁻¹) | (g · mol.⁻¹) | (g · mol.⁻¹) | (g · mol.⁻¹) | (M_{w}/Mₙ)^{b} | level |
| Acetal-PEO | 4400 | — | — | 2980 | 1.051 | 100%^{a} |
| Acetal-PEO-b-PBCL | 4400 | 5000 | 2600 | 6634 | 1.088 | 100%^{a} |
| Aldehyde-PEO-b- | 4400 | 5000 | 1900 | 6634 | 1.738 | 30.1%^{a} |
| PBCL | | | | | | |
| P160-PEO-b-PBCL | 4400 | 5000 | 1900 | 7580 | 1.535 | 20.49%^{c} |
| RGfDK-PEO-b-PBCL | 4400 | 5000 | 1900 | 7580 | 1.15 | 17.39%^{d} |
| P160-RGfKD-b-PBCL | 4400 | 5000 | 1900 | 4068 | 1.101 | 6.15%^{c} |
| | | | | | | 15.65%^{d} |

^{a}Determined by ¹H NMR;
^{b}Determined by GPC;
^{c}Estimated based on conjugation efficiency of P160
^{d}Estimated based on conjugation efficiency of cRGD

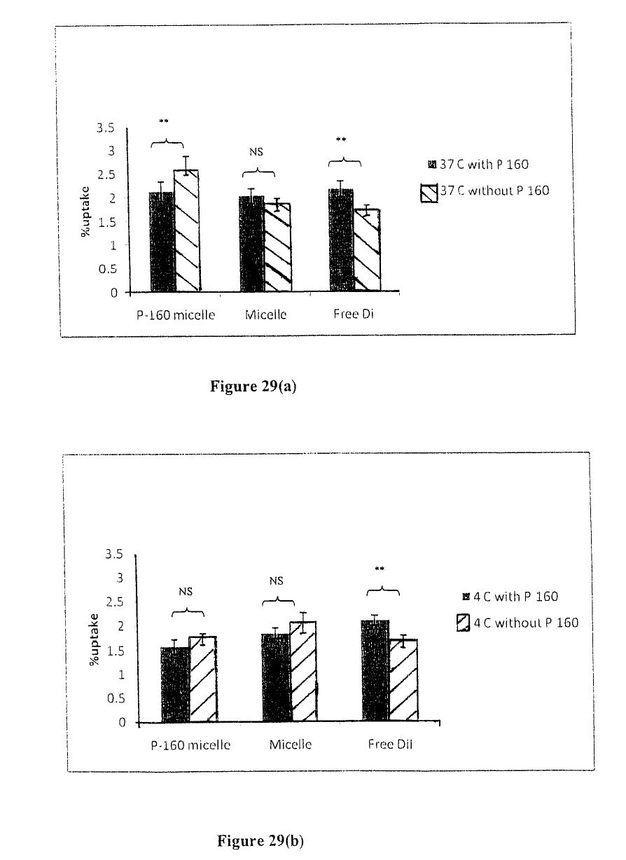

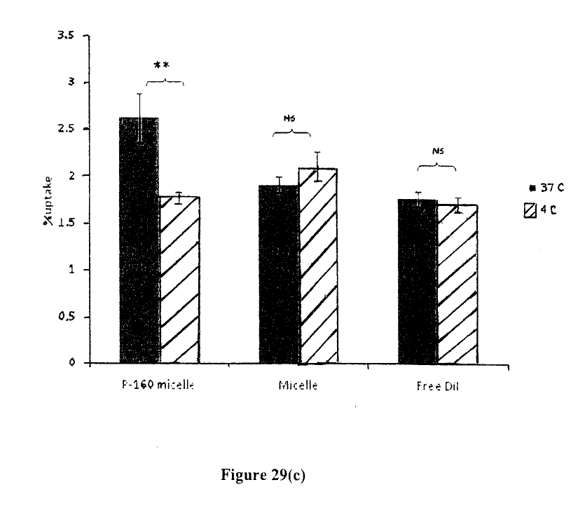

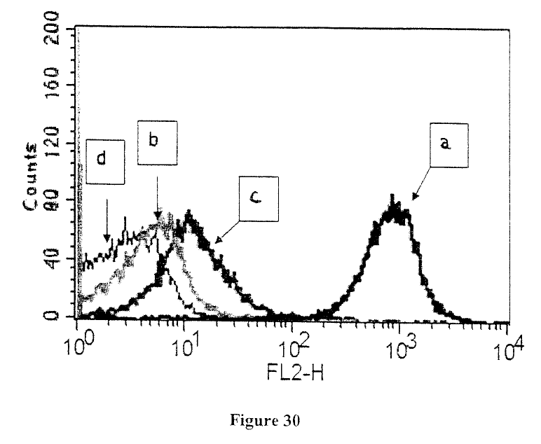

Fluorescence spectroscopy and flow cytometry studies of DiI loaded P-160 micelles on MDA-MB-435 cells showed an increased uptake for the encapsulated DiI in P-160 decorated micelles in comparison to DiI loaded in acetal micelle and free DiI in MDA-MB-435 cells at 37° C. (FIGS. 29-30) but not at 4° C. Moreover, the addition of free P-160 reduced the uptake of P-160 micelles only at 37° C. but didn't affect the uptake of unmodified micelle at both temperatures. This demonstrates the involvement of receptor mediated endocytosis in the uptake of P-160 micelles.

1. A compound of formula I: 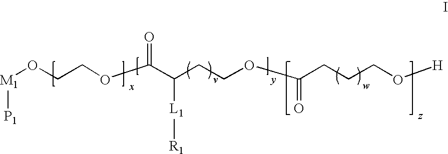
wherein
M₁ is a linker group selected from the group consisting of a single bond, a methyl group, an ethyl group, a propyl group or a C₄₋₁₀alkyl group;
P₁ is CH₃, a reactive functional group or a targeting moiety;
L₁ is a linker group selected from the group consisting of a single bond, —C(O)—O—, —C(O)— and —C(O)NHR²;
R₁ is selected from the group consisting of H, OH, hydrazone, polyamine, polyamine-CF₃, protected polyamine, C₁₋₂₀alkyl, C₃₋₂₀cycloalkyl and aryl, said latter three groups may be optionally substituted and in which one or more of the carbons of the alkyl, cycloalkyl or aryl groups may optionally be replaced with O, S, N, NR² or N(R²)₂ or R₁ is a bioactive agent;
R² is H, NH₂, NH-Fmoc or C₁₋₆alkyl;
v and w are, independently of each other, an integer independently selected from 1 to 4.
x is an integer between 10 and 300;
y is an integer between 5 and 100;
z is an integer between 0 and 100;
wherein aryl is mono- or bicyclic aromatic radical containing from 6 to 14 carbon atoms having a single ring or multiple condensed rings; and
wherein the optional substituents are selected from the group consisting of halo, OH, OC₁₋₆alkyl, C₁₋₆alkyl, C₂₋₆alkenyl, C₂₋₆alkenyloxy, NH₂, NH(C₁₋₆alkyl), N(C₁₋₆alkyl)(C₁₋₆alkyl), CN, NO₂, C(O)C₁₋₆alkyl, C(O)OC₁₋₆alkyl, SO₂C₁₋₆alkyl, SO₂NH₂, SO₂NHC₁₋₆alkyl, phenyl and C₁₋₆alkylenephenyl.

2. A compound as claimed in claim 1, wherein P₁ is a reactive functional group selected from the group consisting of —CH(O—R)₂, where R is a methyl, ethyl, or any other alkyl group; a carbonyl group; an aldehyde; an alcohol; an amino group; a protected amino group; a carboxyl group; a protected carboxyl group; a mercapto group; a protected mercapto group; a hydrazone; a protected hydrazone; or phenyl or phenyl-alkyl group which has a substituent selected from the group consisting of an acetal group, —C(O—R³)—O—R⁴, a carbonyl group, an aldehyde, an alcohol, an amino group, a carboxyl group and a mercapto group on benzene ring, where R³ and R⁴ are H or C₁₋₆alkyl.

3. A compound as claimed in claim 1, wherein P₁ is a reactive functional group selected from the group consisting of hydroxyl, protected hydroxyl, active ester, n-hydroxysuccinimidyl, 1-benzotriazolyl, p-nitrophenyl, imidazolyl esters, active carbonate, n-hydroxysuccinimidyl, 1-benzotriazolyl, p-nitrophenyl, imidazolyl carbonate, acetal, aldehyde, aldehyde hydrates, alkyl or aryl sulfonate, halide, disulfide derivatives, o-pyridyl disulfidyl, alkenyl, acrylate, methacrylate, acrylamide, active sulfone, amine, protected amine, hydrazide, protected hydrazide, thiol, protected thiol, carboxylic acid, protected carboxylic acid, isocyanate, isothiocyanate, maleimide, vinylsulfone, dithiopyridine, vinylpyridine, iodoacetamide, epoxide, glyoxals, diones, mesylates, tosylates, or tresylate.

4. A compound as claimed in claim 1, wherein P₁ is a targeting moiety selected from the group consisting of hydroxyapatite-targeting moieties such as bisphosphonates, polyaspartic acid, polyglutamic acid and aminophosphosugars; proteins; antibodies; antibody fragments; peptides; carbohydrates; lipids; oligonucleotides; DNA; RNA; or small molecules having a molecular weight less than 2000 Daltons.

5. A compound as claimed in claim 2, wherein M₁ is an ethyl group and P₁ is —CH(O—R)₂, where R is a methyl, ethyl, or any other alkyl group.

6. A compound as claimed in claim 5, wherein R is an ethyl group.

7. A compound as claimed in claim 1, wherein M₁ is a propyl group and P₁ is a targeting moiety, whereby the targeting moiety is an integrin ligand.

8. A compound as claimed in claim 7, wherein the integrin ligand is a peptide containing the cell-binding domain Arg-Gly-Asp.

9. A compound as claimed in claim 8, wherein the integrin ligand is Gly-Arg-Gly-Asp-Ser (GRGDS), cyclo(-Arg-Gly-Asp-_{D}-Phe-Lys-) (cRGDfK) or Ala-Cys-Asp-Cys-Arg-Gly-Asp-Cys-Phe-Cys-Gly (RGD4C).

10. A compound as claimed in claim 1, wherein M₁ is a propyl group and P₁ is a targeting moiety, whereby the targeting moiety is a neuroblastoma tumor cell-binding peptide.

11. A compound as claimed in claim 10, wherein the neuroblastoma tumor cell-binding peptide is Val-Pro-Trp-Glu-Pro-Ala-Tyr-Gln-Arg-Phe-Thr (p160).

12. A compound as claimed in claim 1, wherein L₁ is —C(O)—, and R₁ is either —OH or a bioactive agent.

13. A compound as claimed in claim 1, wherein L₁ is —C(O)—O— and R₁ is a benzyl group.

14. A compound as claimed in claim 1, wherein L₁ is —C(O)—NHR² and R² is Fmoc or NH₂.

15. A compound as claimed in claim 12, wherein R₁ is a bioactive agent and the bioactive agent is selected from the group consisting of DNA; other nucleic acid based drugs such as siRNA, oligonucleotides, ribozymes; protein and a drug selected from the group consisting of cucurbitacins, curcumin, resveratrol, buscopan, celecoxib, doxorubicin (DOX), amphotericin B, methotrexate, cisplatin, paclitaxel, etoposide, cyclosporine A, PSC833, amiodarone, rapamycine, camptothecin, cholesterol and ergoesterol, dexamethasone, prednisone, cortisol, testosterone, dromostanolone, testolactone, diethelstilbestrol, ethinyl estradiol, budesonide, beclometasone and vitamin D.

16. A compound as claimed in claim 15, wherein the bioactive agent is doxorubicin (DOX), cholesterol or ergoesterol.

17. A composition comprising a compound of formula I according to claim 1 and a bioactive agent, wherein the compound of formula I forms a micelle around the bioactive agent.

18. The composition according to claim 17, wherein the compound of formula I forms a micelle around the bioactive agent by chemical conjugation, electrostatic complexation and physical encapsulation.

19. The composition according to claim 17, wherein the bioactive agent is selected from the group consisting of DNA, siRNA, RNA, oligonucleotide, ribozymes, protein, peptide and drug.

20. The composition according to claim 19, wherein the bioactive agent is a drug selected from the group consisting of cucurbitacins, curcumin, resveratrol, buscopan, celecoxib, doxorubicin (DOX), amphotericin B, methotrexate, cisplatin, paclitaxel, etoposide, cyclosporine A, PSC833, amiodarone, rapamycine, camptothecin, cholesterol and ergoesterol, dexamethasone, prednisone, cortisol, testosterone, dromostanolone, testolactone, diethelstilbestrol, ethinyl estradiol, budesonide, beclometasone and vitamin D.

21. The composition according to claim 20, wherein the drug is doxorubicin (DOX), cholesterol or ergoesterol.

22. The composition according to claim 21, wherein the drug is doxorubicin (DOX).

23. A method of delivering a bioactive agent to a subject, comprising administering to the subject a compound of formula I according to claim 1 which is capable of forming a micelle around an effective amount of the bioactive agent.

24. The method according to claim 23, wherein the bioactive agent is selected from the group consisting of DNA, RNA, oligonucleotide, protein, peptide and drug.

25. The method according to claim 24, wherein the bioactive agent is a drug and the drug is doxorubicin (DOX), cholesterol or ergoesterol.

26. The compound of formula I as claimed in claim 1, wherein the functionalized esters are randomly distributed throughout the poly(ester) block.

27. The compound of formula I as claimed in claim 1, wherein the functionalized esters are grouped in a block within the poly(ester).
